(19) United States
(12) Patent Application Publication
(10) Pub. No.: US 2023/0263980 A1
(43) Pub. Date: Aug. 24, 2023

(54) PULMONARY ISOLATION, VENTILATION AND TREATMENT APPARATUS AND METHODS FOR USE

(71) Applicant: Stargard Technologies, Inc., Los Angeles, CA (US)

(72) Inventor: Saverio LA FRANCESCA, Los Angeles, CA (US)

(21) Appl. No.: 18/173,047

(22) Filed: Feb. 22, 2023

Related U.S. Application Data
(60) Provisional application No. 63/313,242, filed on Feb. 23, 2022.
(60) Provisional application No. 63/379,024, filed on Oct. 11, 2022.

(51) Int. Cl. A61M 16/04; A61M 16/00; A61B 1/267; A61M 16/14

(52) U.S. Cl. CPC A61M 16/0488; A61M 16/022; A61M 16/0434; A61B 1/2676; A61M 16/14; A61M 2205/587; A61M 2016/0027; A61M 2025/0002

(57) ABSTRACT

A pulmonary isolation and ventilation apparatus (e.g., device, system, etc.) is disclosed. The apparatus may include a controller, a steerable outer member (e.g., outer sleeve body), configured to hold an inner member (e.g., endoscope) and a handpiece. The apparatus may be configured as a sleeve for use with any inner member or the apparatus may include the inner member. Through the handpiece, a user may position the outer sleeve body over an endoscope within a target region of the lungs. The position may be verified by the user using a camera of the endoscope. The endoscope may be removed without disrupting the lungs and the positioning within the lungs. The apparatus may provide improved ventilator functionality to the identified region of the lungs through outer member and may provide for isolating a selective targeted area of the lungs for treatment.

### CLAIM OF PRIORITY

This patent application claims priority to U.S. Provisional Patent Application No. 63/313,242, titled “PULMONARY VISUALIZATION AND VENTILATION APPARATUS AND METHODS FOR USE,” filed on Feb. 23, 2022, and to U.S. Provisional Patent Application No. 63/379,024, titled “PULMONARY ISOLATION AND VENTILATION APPARATUS AND METHODS FOR USE,” and filed on Oct. 11, 2022, each of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

The lungs are one of the largest organs in the body. They are composed of separate areas, including lobes and segments. In general, each lung has 10 segments, as illustrated in FIGS. 15A-15D (showing anterior, posterior, lateral and medical views, respectively). The ascending upper division bronchus may be approximately 7 mm in diameter, and the diameters of the downstream airways may be slightly smaller; for example, the lobular and segmental bronchi may be between 5-8 mm, the subsegmental bronchi and bronchiole may be between 1.5-3 mm, the lobular bronchiole may be about 1 mm, the terminal bronchiole may be about 0.7 mm and the acinar bronchiole may be about 0.5 mm or smaller. Remarkably, estimates of the total surface area of lungs may be as large as 50 to 75 square meters (540 to 810 sq ft). Thus, a significant amount of material (e.g., drug) must be provided to treat the lungs.

Many pulmonary conditions, such as lung cancer or pneumonia (see, e.g., FIGS. 16A-C), affect only some specific areas 1601, 1601′, 1601″, 1601″′ of the lungs and, therefore, treatment should be selective and targeted to these specific areas. However, current techniques do not allow for the selective and targeted insertion of a treatment tube in the peripheral areas under direct endoscopic visualization nor do they allow for the selective isolation of any specific area with the concomitant ability to only deliver treatment to the desired lung areas, while also monitoring the treatment. Isolating a single segment out of the 20 lung segments may reduce the necessary treatment area by at least 19 fold.

Currently positioning of tubes in the lungs may require a guidewire, which may be unreliable for obtaining and maintaining the precise location of the tip of the tube and may require a C-arm (e.g., an X-ray machine) for positioning. It would be beneficial to provide systems and method that can accurately position a tube or scope in the lungs without requiring the use of a guidewire.

In addition, conventional endoscopic systems for the airways do not allow direct visualization or monitoring of airway pressure or temperature downstream of a selected and blocked airway. For example, atelectasis or collapsed lung regions cannot be selectively recruited, since any attempt to do so may deliver air or gases to both lungs in their entirety. Thus, it would be particularly beneficial to isolate the affected area(s) of the lung and monitor downstream pressure in order to avoid overdistention of the airways while obtaining maximal recruitment for the treatment of atelectasis. Conventional systems cannot be used to block a defined area and deliver drugs/gases or any therapeutics or any substances to that defined area. Commercially available pulmonary delivery systems rely on oral or nasal delivery or the delivery through an endotracheal tube, which is far from ideal, because is not selective. Nebulization/delivery through an endotracheal tube delivers the drug/therapeutic gas substance in the trachea first and then into the entire tracheobronchial tree, which makes it difficult or impossible to achieve target agent (e.g., drug agent) concentrations and achieve homogeneous distribution of agents (e.g., drug agents). Current techniques require that the largest part of the inhaled dose be deposited in the healthy adjacent lobes and/or contralateral lung. Concentrating these compounds in the healthy respiratory tract might dramatically increase the severity of these toxicities, as illustrated in FIG. 17.

What is needed are apparatuses and methods for isolated regions of a lung (including relatively small, sub-regions) with sufficient sensing and visualization while providing large-bore access for ventilation and/or treatment.

### SUMMARY OF THE DISCLOSURE

The present invention relates generally to the field of airway management and therapy and more specifically to the field of selective lung ventilation, nebulization, and therapeutic targeted (e.g., local) delivery to treat a variety of pulmonary diseases.

Described herein are apparatuses (e.g., devices, systems, etc.) and methods for delivering pulmonary treatment. The apparatus may be configured as a device that combines visualization, light source, one or more sensors (e.g., pressure sensor, temperature sensor, etc.), large-bore ventilation operations, and agent delivery operations into a portable system. The visualization operations allow a user to precisely target the delivery of pulmonary therapies to small areas or regions of the lungs instead of trying to treat the entirety of the lungs. The apparatus may include an outer, steerable member (outer sleeve body) that includes a pressure sensor, into which an inner, reusable core portion (e.g., endoscope) fits snugly while allowing steering and positioning of the outer sleeve body. The outer sleeve body may include one or more inflatable lumen at the distal end region for isolating a region of the lung. In general, the inner member may be a separate, commercially available endoscope, or it may be a specific member (endoscope) adapted and configured for use with the outer sleeve body. The inner member may fit within the inner lumen of the outer member and may be removed once positioned to provide a large-bore region for access to the isolate lung region. The outer member may include one or more sensors (e.g., pressure sensors). The outer member may be steerable, including steerable with the inner member within the outer member. The inner member may include visualization (e.g., an imaging sensor, optical fibers, etc.) and one or more light sources, and one or more lumen (smaller-bore lumen). The outer diameter of the inner member may be keyed to the inner diameter of the outer member. The inner member may extend to the distal end of the outer member, or in some examples may extend just proximal to the distal end of the outer member, or in some examples may extend distal from the distal end of the outer member. The outer members may be referred to as a multi-functional endobronchial tube. The inner member may be composed of just the steerable components necessary to achieve positioning of the outer member, while the outer component may include some or all the sensors.

In general, as described herein, the pulmonary isolation sleeves described herein may be configured and/or adapted for use with any endoscope, including re-usable endoscope or single-use endoscopes. In some examples the size of the lumen may be set to be approximately the same size (same inner diameter, ID) as the outer diameter (OD) of the endoscope with which it is to be used. Different sizes (small, medium, large, etc.) having different ODs configured to correspond to commercially available endoscopes may be used. Alternatively, in any of these methods and apparatuses the inner member may be part of the apparatus. For example, the outer member (“outer elongate body”) may be configured as an outer sleeve, having an elongate, flexible (and in some examples, steerable) outer sleeve body. Thus, the inner member (e.g., endoscope) may be separate from the apparatus. However, in any of the methods and apparatuses described herein the inner member may be part of the apparatus (e.g., system).

In general, the apparatuses described herein may be referred to as pulmonary isolation system or as pulmonary isolation sleeves (or may include pulmonary isolation sleeves). For example, a pulmonary isolation sleeve may be configured for use with an endoscope. As mentioned, this endoscope may be provided by a third party or may be part of the system. For example, a pulmonary isolation sleeve system may include: an outer sleeve body comprising a flexible endobronchial tube having a lumen extending therethrough; a pressure sensor at a distal end face of the outer sleeve body; wherein the lumen is configured to hold the endoscope therein so that the endoscope may rotate relative to the outer sleeve; a first handpiece portion coupled to the outer sleeve; and a controller configured to couple to the first handpiece portion and to receive input from the pressure sensor, and to couple to the second handpiece portion.

In some examples the outer sleeve may be configured to be secured in place in the desired lung region for a certain period of time, i.e. hours, days or weeks in order to repeat the therapeutics maneuvers to a patient through the lumen of the outer sleeve body that was previously occupied by the elongate tube (inner sleeve). This may be achieved by including an outer retaining element that forms a seal with the wall or walls of the region of the lung. Because it may be necessary to remain in position against air pressure, the outer (sealing) element may be configured to robustly seal within the lungs without irritating or damaging the lung region, as described herein.

In some examples, the outer sleeve may be configured to allow for selecting and isolating, under visualization, in order to deliver energy as in radiofrequency ablation (RFA), microwave ablation (MWA), cryoablation (CRA) and photodynamic therapy, either directly through the outer sleeve or via of other devices, i.e. catheters inserted in the channel.

The controller may be further configured to regulate a pressure at the distal end of the outer sleeve body when the inner sleeve of an endoscope is withdrawn from the lumen of the outer sleeve body. In some examples the controller is configured to apply respiration through the lumen when the inner sleeve or an endoscope is removed from the lumen.

In any of these apparatuses the controller may be configured to monitor any of the sensors and use sensed data for feedback and/or control of the apparatus. For example, the controller may be configured to monitor the temperature at the distal end of the outer sleeve body when the endoscope is withdrawn from the lumen of the outer sleeve body.

Any of these apparatuses may include a nebulizer configured to administer a therapeutic from the outer sleeve body.

Any of these apparatuses may include an anchor for anchoring the apparatus (e.g., the outer sleeve body) within the lungs. For example, any of these apparatuses may include an expandable anchor, such as an inflatable anchor and/or a mechanically expandable/collapsible (e.g., stent-like) anchor, at a distal end region of the outer sleeve body.

The controller may be configured to apply positive or negative pressure through a pressure channel in the outer sleeve body to regulate the pressure at the distal end of the outer sleeve body when the endoscope is withdrawn from the lumen of the outer sleeve body.

In any of these examples the outer sleeve body may be steerable.

The controller may be configured to operate independently of the endoscope. For example, the controller may be configured to display information (e.g., pressure, respiration, etc.) separately from and/or independently of, the endoscope, including a controller (e.g., “tower”) for the endoscope. Alternatively, any of these methods and apparatuses described herein may be configured so that the pulmonary isolation sleeve controller communicates with (or in some examples, integrates with) the endoscope and/or endoscope controller.

The apparatuses described herein may be configured to provide pulmonary isolation and ventilation system. The apparatus may include a controller, a multi-function endobronchial tube (outer tube), an inner member, and a handpiece coupled to the controller and the multi-function endobronchial tube. The handpiece may include a ventilation handle configured to control, at least in part, ventilator operations that provide air for isolated lung regions through the multi-function endobronchial tube and a visualization handle configured to control, at least in part, visualization operations that display images captured through a distal end of the multi-function endobronchial tube and/or the inner tube. In some examples the visualization handle is configured to be removably coupled to the ventilation handle. In some examples the handpiece may be a two-part handpiece; one part coupled to the inner member, the other part coupled to the outer member (e.g., the multi-functional endobronchial tube).

For example, the visualization handle may be coupled to the inner member, e.g., an elongate tube that is configured to fit within the multi-function endobronchial tube. Thus, the elongate tube may be removably coupled to the multi-function endobronchial tube. Further, in some examples the elongate tube may be configured to be removed from the multi-function endobronchial tube.

In some examples, the distal end of the outer sleeve may include one or more sensors (including duplicate sensors that may be individually or collectively controlled and/or analyzed). For example, the sensors may include one or more cameras to capture images (stills, video, etc.), a light source for illumination, a pressure sensor for distal airway pressure monitoring, a temperature sensor or a sensor that capture a specific kind of energy, etc. For example, sensors may include thermal, magnetic, light, X-rays or proton beam sensing sensors. In some examples the inner sleeve may be removable, and all (or most) of the sensors may be included in the outer sleeve.

In some examples, the elongate tube (e.g., inner member) may include at least one light source configured to provide light to a distal end of the elongate tube. Furthermore, in some examples the multi-function endobronchial tube (e.g., outer member) may be configured to provide gases to the isolated lung regions after the elongate tube is removed.

The inner member (elongate tube) may include any feasible number of channels or lumen. For example, the inner member may include one or more lumen to provide negative pressure, liquid for irrigation, or a combination thereof. In some examples. The inner member may include a camera configured to capture images at the distal end of the multi-function endobronchial tube.

In some examples, the distal end of the outer sleeve may include any feasible numbers of sensors, such as, but not limited to, a camera to capture images and/or a light source for illumination, a pressure sensor for distal airway pressure monitoring, a temperature sensor or a sensor that capture a specific kind of energy, e.g., thermal, magnetic, light, X-rays or proton beam. In some examples the inner member (endoscope, also referred to herein as an inner sleeve) may be removed, and all or a majority of the sensors may be included in the outer sleeve.

In some examples, a proximal end of the multi-function endobronchial tube (outer member) may be coupled to the ventilation handle and a distal end of the multi-function endobronchial tube may include a pressure sensor. In some applications, the controller may be configured to provide air to the isolated lung regions based, at least in part, on pressure determined by the pressure sensor.

In some examples, the multi-function endobronchial tube may include a camera configured to provide image data to the controller. Furthermore, the controller may include a display configured to display images from the camera. In some variations, the system may include a nebulizer configured to administer a liquid or gas therapeutic agent from the ventilation handle and out of the outer member, including out through the central bore of the outer member, the channel in which the inner member resides. Thus, the ventilation handle may include a port configured to receive liquid or gas therapeutic agents.

In some examples, the system may include an inflatable occlusion member coupled proximate to the distal end of the multi-function endobronchial tube. Furthermore, in some examples the ventilation handle may include a valve configured to provide an air-tight seal when the visualization handle is separated from the ventilation handle.

Also described herein are methods of treating a patient by isolating a region of the lungs using an apparatus as described herein. For example, a method may include: inserting an endoscope within a lumen of a flexible outer sleeve body of a pulmonary isolation sleeve into a region of a patient's lung, wherein the endoscope is within the lumen of the outer sleeve body and extends to a distal end region of the outer sleeve body; confirming placement of a distal end of the outer sleeve body withing the lung; anchoring the distal end of the outer sleeve body within the lung; withdraw the endoscope from the lumen of the outer sleeve body; delivering a nebulized agent to a targeted region of the lung through the lumen of the outer sleeve body; and ventilating a patient through the lumen of the outer sleeve body that was previously occupied by the elongate tube.

Any of these methods may include controlling the application of pressure through the outer sleeve body to regulate a pressure at the distal end of the outer sleeve body as the endoscope is withdrawn from the lumen of the outer sleeve body. Controlling pressure may include preventing a vacuum at the distal end of the outer sleeve body. In some examples controlling pressure comprises maintaining the pressure within a target range of positive pressure.

Also described herein are methods of treating a patient by isolating a region of the lungs using an apparatus as described herein. For example, a method may include: inserting an endoscope within a lumen of a flexible outer sleeve (which may be referred to herein in any of these examples as a pulmonary isolation sleeve) into a region of a patient's lung. The pulmonary isolation sleeve may be inserted into the lung of the patient. The endoscope may be positioned within the lumen of the outer sleeve body and may extend to a distal end region of the outer sleeve body; confirming placement of a distal end of the outer sleeve body withing the lung. Any of these methods may include anchoring and/or sealing the distal end of the outer sleeve body (pulmonary isolation sleeve) within the lung (e.g., lung segment). The endoscope may then be withdrawn from the lumen of the outer sleeve body. Once the sealing and/or anchoring mechanism is deployed, the selected area of the lung (e.g., segment or sub-set of segments) may be isolated, and an agent may be delivered. In some examples the agent is a hydrogel material that can be delivered without its deposition being altered by ventilation. The hydrogel can have the time to adhere to the airways.

In any of these methods inserting comprises steering the outer sleeve body.

In any of these methods confirming placement may comprise visualizing the region of the lung near the distal end of the outer sleeve body using one or more cameras of the endoscope.

Anchoring may comprise inflating an inflatable balloon at the distal end of the outer sleeve body.

Any of these methods may include disengaging a first handle (first handle portion) that is coupled to the endoscope from a second handle (e.g., second handle portion) that is coupled to the outer sleeve body prior to withdrawing the endoscope from the lumen of the outer sleeve body (and therefore an of these methods may include coupling the first handle portion to the second handle portion). A handle may be equivalently referred to as a handpiece.

As described in greater detail here, any of the methods described herein may include applying an atomized drug from a distal end of the outer sleeve body.

In some examples the method may include inserting, via a handpiece including visualization handle coupled to a ventilation handle, a multi-function endobronchial tube concentrically positioned around an inner member, into a patient's trachea, confirming placement of a distal end of the multi-function endobronchial tube with a images from a camera proximate to the distal end of the multi-function endobronchial tube and/or a pressure sensor on the outer member, anchoring the distal end of the multi-function endobronchial tube (e.g., by inflating a balloon), removing the inner member including the visualization assembly that from within a large-bore channel of the multi-function endobronchial tube (outer tube), and ventilating a patient through the large-bore channel in the multi-function endobronchial tube that was previously occupied by the elongate tube.

In general, the apparatuses described herein may include an anchor for anchoring the outer member within the body, e.g., within a bronchial passage, at the distal end. Any appropriate anchor may be used, including an inflatable anchor (e.g., one or more balloons, including an annular balloon), a mesh basket, expandable gasket, spring element, one or more arms or leaflets extendable/retractable from the outer surface, etc. In general, the anchor may be deployed by expanding radially outwards, and removed by retracting inwards.

In some examples, removing the visualization assembly may include separating the visualization handle from the ventilation handle, and withdrawing, from the multi-function endobronchial tube, the elongate tube coupled to the visualization handle. The step of removing the visualization assembly may be performed without disturbing the position of the outer member within the lungs, by separating the inner member from the outer member. The inner member may be configured to normalize the pressure within the distal end region of the lung as the inner member is withdrawn proximally from the large bore of the outer member. This may prevent forming a negative pressure as the plunger-like inner member is withdrawn proximally, which may otherwise disrupt and possibly damage the tissue. For example, the inner member may include one or more lumen or channels into which air is added (pumped) when removing the inner member. One or more pressure sensors on the outer member may monitor the pressure within the isolated region of the lungs and/or at the distal end of the outer member and may automatically regulate the applied pressure and/or withdrawal rate of the inner member. For example, the controller of the apparatus may be configured to control pressure at the distal end region of the outer member as the inner member is withdrawn proximally, prior to and during withdrawal of the inner member. This may be referred to as withdrawal control logic.

In some variations, anchoring may include inflating an occlusion member disposed proximate to the distal end of the multi-function endobronchial tube.

In some examples, confirming placement may include displaying the images from the camera on a display of a controller coupled to the handpiece.

As mentioned, any of these methods may further include determining and/or monitoring a pressure at the distal end of the multi-function endobronchial tube and ventilating the patient based at least in part on the determined pressure.

Any of the methods and apparatuses described herein may be configured for robotic operation. For example, any of the methods described herein may be performed robotically or as part of a robotic system, including navigation and operation of a robotic element (e.g., robotic arm). Any of the steps of the methods described herein, including the steps of inserting the outer member, confirming placement of a distal end of the outer member, anchoring the distal end of the outer member within the lung, withdraw the elongate inner member from the lumen of the outer member, and/or delivering a nebulized (or any other) agent to a targeted region of the lung through the lumen of the outer member may be performed robotically using one or more robotically controlled or operated members. For example, any of the steps of inserting an endoscope, confirming placement of the outer sleeve body withing the lung, anchoring the distal end of the outer sleeve body within the lung, withdraw the endoscope from the lumen of the outer sleeve body and/or delivering a nebulized agent to a targeted region of the lung through the lumen of the outer sleeve body may be performed robotically.

Further, any of the apparatuses described herein may include a robotic arm and/or the apparatuses described herein may be configured for robotic operation. For example, the pulmonary isolation and ventilation system may include an outer member that is adapted for robotic user, and/or an elongate inner member that is adapted for robotic use, and/or a first handpiece portion coupled to the outer member adapted for robotic use, and/or a second handpiece portion coupled to the inner member adapted for robotic use. As used here adaptations for robotic use may include one or more attachment regions for coupling to a robot and/or connectors for connecting one or more portions of the system to the controller operating the robot. In some cases the controller configured to couple to the first handpiece portion and to receive input from the pressure sensor, and to couple to the second handpiece portion may also be configured to communicate with the robotic sub-system.

For example, described herein are pulmonary isolation and ventilation systems comprising: an outer member configured as a sheath comprising a flexible endobronchial tube having a lumen extending therethrough and a one or more sensors at a distal end, the outer member comprising an elongate inner lumen extending from the distal end to a proximal end, and a sealing anchor configured to extend from an outer diameter of a distal end region of the outer member to seal the outer member within a bronchial lumen; and a handpiece portion coupled to the outer member; and a controller configured to couple to the handpiece portion and to receive input from the one or more sensors.

The elongate inner lumen may comprise 50% or more (e.g., 55% or more, 60% or more, 65% or more, 70% or more, 75% or more, 80% or more, etc.) of the cross-sectional area of the outer member, e.g., at the distal end of the device. The cross-sectional area may exclude the expandable (e.g., inflatable, etc.) sealing member.

Any of these systems may include an elongate inner member configured to fit within the elongate inner lumen and to engage the handpiece. The controller may be further configured to regulate a pressure at the distal end of the outer member. In some examples the one or more sensors may comprise one or more cameras and wherein the outer member comprises one or more light sources. In some examples the one or more sensors may comprise one or more of: visualization sensors, illumination sensors, temperature sensors, or pressure sensors.

Any appropriate sealing anchor may be used. In some examples the sealing anchor may comprise an expandable balloon. The controller may be configured to apply control the sealing of the sealing anchor (e.g., to expand and/or contract the sealing anchor).

Any of these apparatuses may include a reservoir for the agent to be applied. In some example, the apparatus may include a nebulizer configured to administer an agent from the lumen of the outer member. In some examples the apparatus may include a respirator to apply respiration through the lumen.

Also described herein are methods of using any of these apparatuses. For example, also described herein a methods comprising: inserting an outer member comprising a flexible endobronchial tube having a lumen extending therethrough into a region of a patient's lung, wherein an elongate inner member is sealed within a lumen of the outer member and extends to a distal end region of the outer member; confirming placement of a distal end of the outer member withing the lung; sealingly anchoring the distal end of the outer member within the lung to form a sealed-off region of the lung; withdraw the elongate inner member from the lumen of the outer member; and delivering an agent comprising one or more of: a drug, a hydrogel, a gel, particles, nanoparticles, liposomes, cells, stem cells, vesicles, or gene therapy through the lumen and into the sealed-off region of the lung.

Sealing anchoring the distal end of the outer member may comprise controlling the application of a pressure to expand a sealing anchor from an outer diameter of the outer member. Any of these methods may include feedback from one or more pressure sensors, including a pressure sensor at a distal end of the outer member to indicate that the seal is secured. In some examples, the feedback control may be received from a pressure sensor inflating or expanding the sealing anchor.

Any of these method may include controlling the application of pressure to regulate a pressure at the distal end of the outer member as the elongate inner member is withdrawn from the lumen of the outer member. In any of these methods, controlling pressure may comprise preventing a vacuum at the distal end of the outer member.

As mentioned above, confirming placement may comprise visualizing the region of the lung near the distal end of the outer member using one or more cameras of the inner member.

These methods may further include disengaging a first handle portion that is coupled to the inner member from a second handle portion that is coupled to the outer member prior to withdrawing the inner member from the lumen of the outer member, and/or applying an atomized drug from a distal end of the outer or inner member.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1A shows an example pulmonary isolation and ventilation apparatus, including an inner member and an outer member.

FIG. 1B shows an example of a pulmonary isolation and ventilation apparatus, including an outer member configured as a sleeve for use with any inner member (e.g., endoscope).

FIG. 1C shows the apparatus of FIG. 1B inserted into a lung.

FIG. 2 shows an example of intubation of a patent using an apparatus as described herein.

FIG. 3 shows a simplified diagram of a pulmonary isolation and ventilation apparatus operating in a first (visualization) mode.

FIGS. 4A and 4B show simplified diagrams a pulmonary isolation and ventilation apparatus being used with a patient's lung.

FIG. 5 shows a simplified diagram of a pulmonary isolation and ventilation apparatus operating in a second (ventilation) mode.

FIG. 6A shows an end view of an example multi-function endobronchial tube (outer member) in which an inner member is positioned.

FIG. 6B shows the end view of the example multi-function endobronchial tube of FIG. 6A after the inner member (elongate tube) has been removed from the large bore of the outer member.

FIG. 6C shows a perspective view of an end of one example of an apparatus as described herein, including a multi-function endobronchial tube (outer member) and inner member as shown in FIGS. 6A and 6B.

FIG. 7 shows an example of a distal end of an apparatus including an outer member (multi-function endobronchial tube) including an inner member, in situ.

FIG. 8A shows an end view of an example of an outer member (e.g., multi-function endobronchial tube) and inner member.

FIG. 8B shows the end view of the outer member, e.g., multi-function endobronchial tube, of FIG. 8A after the inner member (e.g., elongate tube) has been removed from the large bore channel (e.g., a second channel) of the outer member.

FIG. 9 shows a simplified diagram of an example of a pulmonary isolation and ventilation apparatus operating in the second (ventilation) mode.

FIG. 10 schematically illustrates an example of a method for operating a pulmonary isolation and ventilation apparatus.

FIG. 11 shows a block diagram of an example of a controller for an apparatus as described herein.

FIGS. 12A-12J illustrate an example of an apparatus and method of use as described herein. FIG. 12A shows an example of a lung that is partially collapsed on one side (left lung), which may be treated with an apparatus as described herein. FIG. 12B is an example of a portion of an apparatus (including a controller) as described herein. FIGS. 12C-12D illustrate placement of an apparatus as described herein within the lungs. FIGS. 12E-12F illustrate anchoring of the distal end of the apparatus at a target region of the lungs. FIGS. 12G-12H illustrate removal of an inner member leaving an outer member of the apparatus having a large bore for the application of respiration and/or active agent(s). FIGS. 121-12J illustrate use of the apparatus to restore a collapsed region of the lung.

FIGS. 13A-13B illustrate an example of an apparatus and method of use as described herein. In FIG. 13A the apparatus (configured as a pulmonary isolation sleeve) is shown inserted an anchored into a branch of a lung (a portion of which is shown removed to visualize) with an endoscope held within the lumen of the apparatus. FIG. 13B shows the apparatus with the endoscope removed.

FIGS. 13C and 13D illustrate an example of an apparatus and method of use as described herein. In FIG. 13C the apparatus (configured as a pulmonary isolation sleeve) is shown inserted an anchored into a branch of a lung (a portion of which is shown removed to visualize) with an endoscope held within the lumen of the apparatus. FIG. 13D shows the apparatus with the endoscope removed.

FIG. 14 illustrates an example in which multiple pulmonary isolation sleeves are shown inserted and anchored into the various regions of the lungs.

FIGS. 15A-15D illustrate the different segments of the lungs. FIG. 15A shows an anterior view, FIG. 15B shows a posterior view, FIGS. 15C and 15D show lateral and medial views, respectively, of an example of a lung.

FIGS. 16A-16C show examples of scans of lungs having localized lung diseases that may be treated with the apparatuses described herein. FIG. 16A shows an example of a lung having two regions of cancer. FIGS. 16B and 16C show examples of pneumonia localized to a region of a lung.

FIG. 17 schematically illustrates how pulmonary delivery routes are not selective, do not target a specific area of the lungs but deliver an agent (e.g., drug) to the entire respiratory tree.

FIG. 18 illustrates an example of a right lower lobar pneumonia with one example of an apparatus as described herein inserted into the selected sub-region of the lungs.

### DETAILED DESCRIPTION

Described herein are apparatuses including modular visualization and ventilation components. These apparatuses and method may enable selective visualization and isolation of bronchial areas and also provide ventilation and drug delivery functions. By combining these functions, pulmonary therapies may be precisely targeted and delivered without the need for separate imaging equipment such as X-ray machines, CT scan systems, ultrasound machines, or the like.

The system may include an outer member, e.g., configured as a multi-function endobronchial tube or sleeve body that includes a pressure sensor located at a distal end (e.g. at a distal-facing end), and one or more outer members that may fit within a main lumen, also referred to herein as a large or primary bore, of the outer member. The pressure sensor may be used to control air pressure that may be used to ventilate a patient and/or to control pressure when inserting/removing components such as the inner member. The pressure sensor may be configured to detect pressure within the region of the lung(s) distal to the end of the sleeve. The outer member (sleeve body) may include a large bore and/or one or more channels or lumens to provide suction (negative pressure) or irrigation to the patient. The large bore of the multi-function endobronchial tube may alternately engage with or carry the inner member, which may itself include or engage with visualization elements (e.g., camera, lights, and the like) or provide air and/or therapeutics to a treatment region.

The apparatuses described herein may include a camera that captures images from a distal end of the apparatus. The user may use the images from the camera to ensure that the multi-function endobronchial cable is positioned in the correct region to receive pulmonary therapy, to partition off a region of the lungs and/or to apply ventilation and/or drugs (e.g., aerosolized agents). The camera may be on the outer and/or inner members.

The apparatus may also include a controller that outputs to a display, such as a screen or touch screen. The display may be used to display ventilator settings as well as images from the camera. Additionally, the apparatus may include a nebulizer to deliver therapeutic agents to the treatment region.

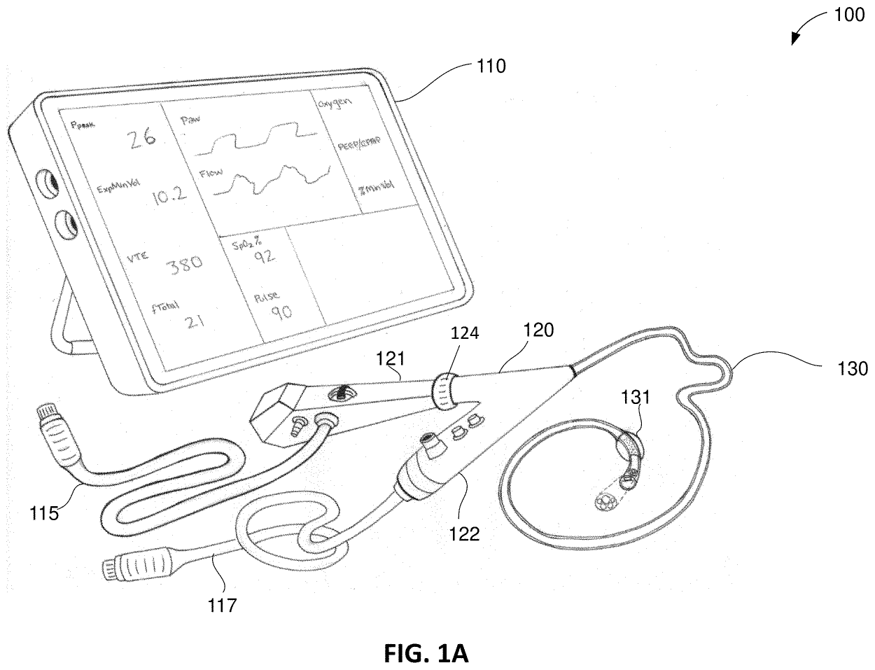

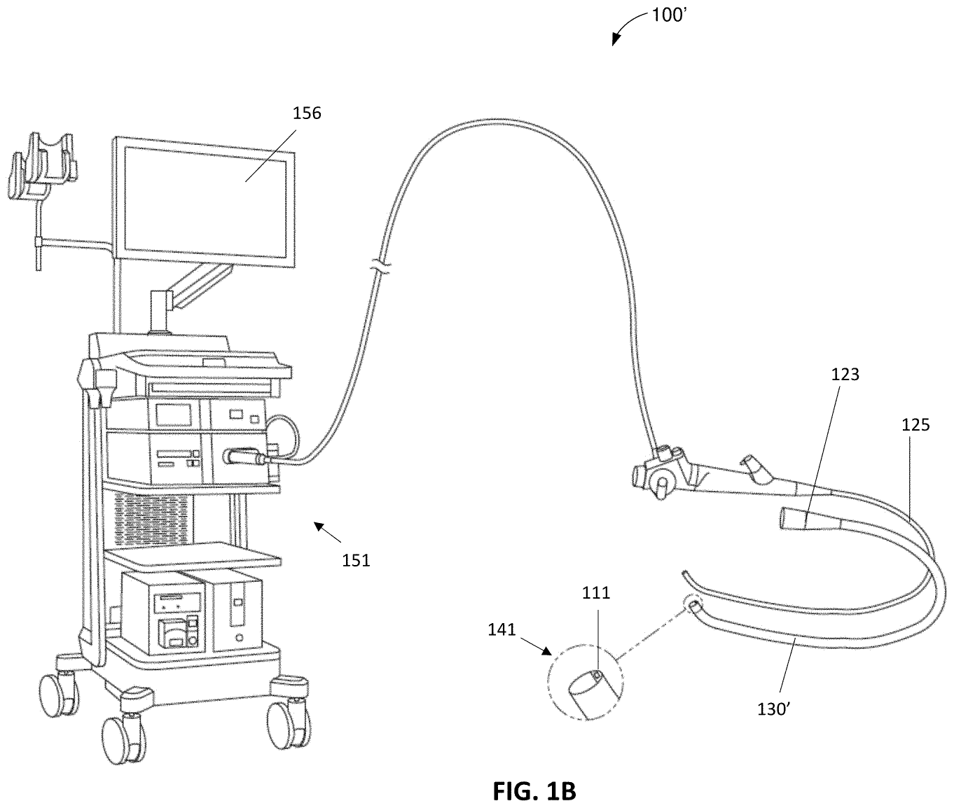

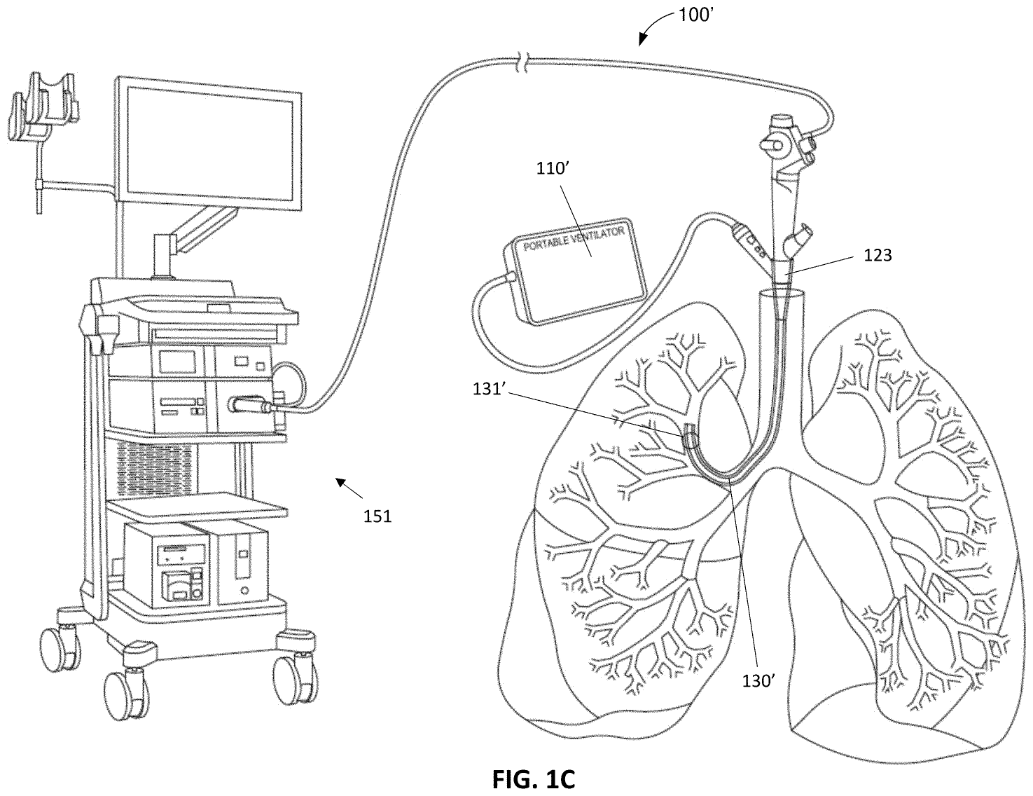

FIG. 1A shows an example of an apparatus configured as a pulmonary isolation and ventilation system 100. The pulmonary isolation and ventilation system 100 may include a controller 110 (including in this example, a touchscreen), a combination handpiece 120, a first cable assembly 115, a second cable assembly 117, and a multi-function endobronchial tube 130 (e.g., outer member). An inner member is also present within the outer member but is not visible in FIG. 1A. The pulmonary isolation and ventilation system 100 may operate in one of at least two modes. In a first (e.g., visualization) mode, pulmonary images from a camera within or coupled to the multi-function endobronchial tube 130 may be displayed by the controller 110. Using the displayed images, a user (e.g., a doctor or other clinician, technician, etc.) may guide a distal end of the endobronchial tube into a target area of the lungs. Thus, the camera and display, including one or more pressure sensors, may be used to confirm and verify the position of the multi-functional endobronchial tube 130. Next, an inflatable occlusion member 131 attached or coupled proximate to a distal end of the multi-function endobronchial tube 130 may be expanded (e.g., inflated) to anchor the multi-function endobronchial tube 130. As mentioned above, other anchors, not limited to balloons, may be used instead of or in addition to an inflatable member.

In this example, the second member including visualization elements (camera, lights, etc.) and/or other sensors may be formed within the inner member. One or more channels (e.g., for applying positive or negative pressure, e.g., air) may also be included. The inner member may be coupled to the handle and may be removed or inserted from the outer member. When inserted or removed, the controller 110 (or a separate controller) may apply positive or negative pressure through one or more channels to maintain the pressure, which may be sensed by the pressure sensor at the distal end region of the outer member, within a target range, which may be automatically set or set to within a predetermined value.

The pulmonary isolation and ventilation system 100 may then operate in a second (ventilation) mode. In the second mode, the inner member including the visualization components (camera, fiber optics, etc.) may be withdrawn from the multi-function endobronchial tube 130, thereby providing a lumen or channel that may be used to introduce air, gases, and/or any feasible therapeutic agents into a bronchial area distal to the end of the multi-function endobronchial tube 130. The apparatus may regulate the pressure (e.g., by applying positive pressure, e.g., air) through a channel as the inner member is withdrawn, which may prevent the creation of potentially harmful negative pressure within the region of the lung at the distal end of the apparatus.

In general, the controller 110 may support operations of the pulmonary isolation and ventilation system 100 in both the first and second operation modes. For example, in the first (visualization) mode, the controller 110 may display camera images (e.g., image data) as well as provide visualization controls (lighting controls, camera controls, inflation controls, and the like) to enable the user to locate and anchor the multi-function endobronchial tube 130 in an affected area of the patient. In the second (ventilation) mode, the controller 110 may display ventilation controls (flow rate, patient vital statistics, and the like) to enable the user to perform ventilator operations for the patient. In some variations, the controller 110 may enable administration of gases and/or therapeutic agents to the patient.

The combination handpiece 120 may be coupled to the controller 110 via a first cable assembly 115 and a second cable assembly 117. The combination handpiece 120 may be a multi-function handpiece that includes a visualization handle 121 portion that is removably coupled to a ventilation handle 122. The visualization handle 121 may include some visualization controls that may be used in conjunction with, or instead of any visualization controls displayed on the controller 110 and/or pressure input (for applying positive and/or negative pressure). Similarly, the ventilation handle 122 may include ventilator controls that may be used in conjunction with, or instead of any ventilator controls displayed on the controller 110. Thus, the visualization handle 121 may be used to control visualization operations and the ventilator handle 122 may be used to control ventilation operations, particularly after the inner member has been removed. The same pressure/ventilation source may be used and coupled to both the inner and outer members, or separate and distinct pressure/ventilation sources may be used.

When operating in the first mode, the user may guide the multi-function endobronchial tube 130 into bronchial passages using the combination handpiece 120. In some embodiments, the combination handpiece 120 may use one or more guide wires to manipulate and guide the multi-function endobronchial tube 130. In some variations, the distal end of the multi-function endobronchial tube 130 can be steered. Some typical distal end movements are 180 degrees up and down (e.g., 180 degrees up and 180 degrees down, 150° up and 130° down, or 180° up and 130° down, etc.). A display on the controller 110 may relay image information to the user so that the user can correctly locate the distal end of the multi-function endobronchial tube 130 with respect to the patient.

The outer member 130 and/or inner member(s) may be steerable. Any appropriate steering element may be used, including one or more wires or tendons for deflecting the distal end of the outer member. For example, the apparatus may include a control 124 (e.g., knob, button, slider, etc.) to control deflection and to steer the apparatus.

When operating in the second mode, the user may separate the visualization handle 121 from the ventilation handle 122 and, in some cases, withdraw an elongate tube (not shown) from a channel or lumen within the multi-function endobronchial tube 130 that includes visualization components. The user may then administer gases and/or medicines to the patient through the ventilation handle 122 and through the channel or lumen in the multi-function endobronchial tube that previously included the visualization components (e.g., the elongate tube). For example, the pulmonary isolation and ventilation system 100 may function as a ventilator to provide air through the multi-function endobronchial tube 130 to the patient.

FIG. 1B shows another example of an apparatus configured as a pulmonary isolation and ventilation system 100′. The pulmonary isolation and ventilation system 100′ (e.g., pulmonary isolation sleeve) may include a controller 110 (not shown in this example), a handpiece 123 (or first handpiece portion), an outer sleeve body 130′ and a distal end region 141 (shown in an enlarged view) including a pressure sensor 111. In this example, the apparatus may also be coupled (wirelessly or wired) to a controller and/or display (not shown).

In FIG. 1B the inner member is an endoscope 151, which may be a commercially available endoscope is shown coupled to an endoscope controller, shown as a tower 155 that includes a display 156. For example, the endoscope may be a bronchoscope (e.g., an Olympus Exera BP-P160) and may be used with a corresponding controller (e.g., Olympus CV-160 Video Processor).

The pulmonary isolation and ventilation system 100′ may engage with the endoscope, e.g., by sliding over the endoscope, and may operate in one of at least two modes. In a first (e.g., visualization) mode, pulmonary images from a camera within or coupled to either the outer member (e.g., sleeve 130′) or the inner member (e.g., endoscope 125) may be displayed by the controller 110. Using the displayed images, a user (e.g., a doctor or other clinician, technician, etc.) may guide a distal end of the sleeve and bronchoscope/endoscope into a target area of the lungs (as shown in FIG. 1C). Thus, the camera and display, including one or more pressure sensors, may be used to confirm and verify the position of the apparatus 100′. In some examples, an anchor (e.g., an inflatable occlusion member 131′) attached or coupled proximate to a distal end of the outer member 130′ may be expanded (e.g., inflated) to anchor the outer member 130′. As mentioned above, other anchors, not limited to balloons, may be used instead of or in addition to an inflatable member.

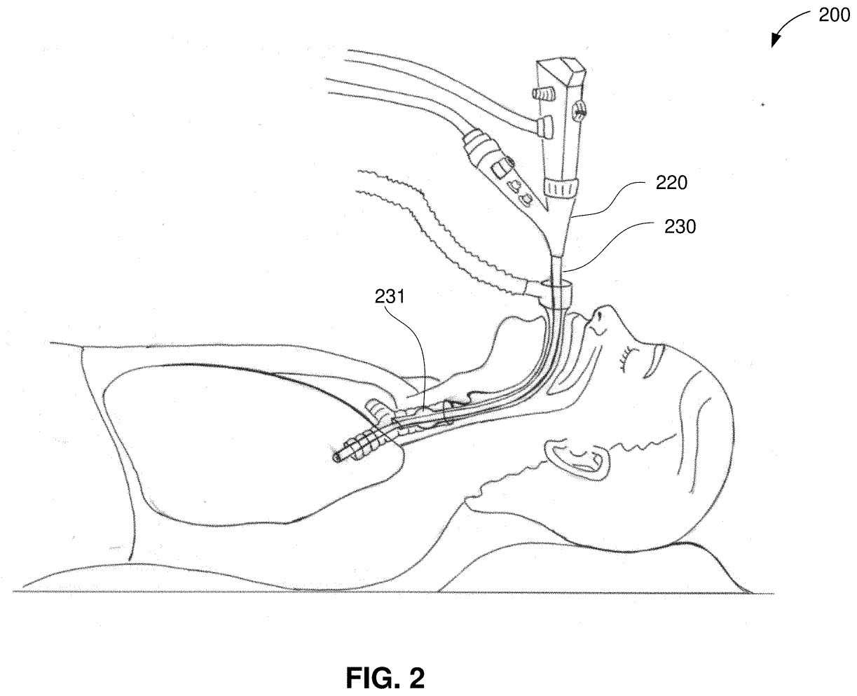

FIG. 2 shows an example of an intubation using an apparatus as described herein 200. As shown, a combination handpiece 220 may be used to guide and insert a multi-function endobronchial tube 230 (outer member) into a patient's airway. The combination handpiece 220 and the apparatus may coordinate operation of both the outer member, multi-function endobronchial tube 230, and the inner member (not shown), and in this example are similar to the combination handpiece 120 and the multi-function endobronchial tube 130 of FIG. 1A. Also shown, an inflatable occlusion member 231 may be inflated to anchor a distal end of the multi-function endobronchial tube 230. Any feasible gas (air, oxygen, etc.) or liquid (water, saline, etc.) may be used to inflate the inflatable occlusion member 231.

In some variations, the inflatable occlusion member 231 may create an isolated treatment area. For example, only portions of a bronchial area that are distal of the inflatable occlusion member 231 only receive ventilation treatment. In one exemplary, non-limited application, the targeted or selective treatment provided herein may advantageously treat localized atelectasis. In some embodiments, donated lungs may receive such selective treatment and transform lungs that were not deemed suitable for transplantation into lungs that are suitable for transplantation.

In some other variations, the inflatable occlusion member 231 may be used to limit a treatment that receives therapeutic agents. For example, gas or liquid therapeutic agents may be introduced into the combination handpiece and directed to the region distal of the multi-function endobronchial tube 230.

FIG. 3 shows an example of an apparatus configured as a pulmonary isolation and ventilation system 300 operating in a first (visualization) mode. The pulmonary isolation and ventilation system 300 may be an example of the pulmonary isolation and ventilation system 100 of FIG. 1A. The pulmonary isolation and ventilation system 300 may include a controller 310, a combination handpiece 320, and a multi-function endobronchial tube 330 as well as an inner member (not visible in FIG. 3). As shown, the multi-function endobronchial tube 330 may be inserted into a patient's trachea and/or lung 350. Using the visualization functions provided by an included camera, the multi-function endobronchial tube 330, and the controller 310, the user may target and isolate specific regions and/or lobes of the patient's lungs 350 to receive treatment, after being steered into position. For example, the user may ventilate or provide therapeutic agents to identified regions of the lungs 350. In some embodiments, an inflatable occlusion member 331 may be used to anchor the multi-function endobronchial tube 330 as well as isolate a region of the lungs. In this manner, any ventilation or therapeutic agents may be provided to a subset of the patient's lungs 350.

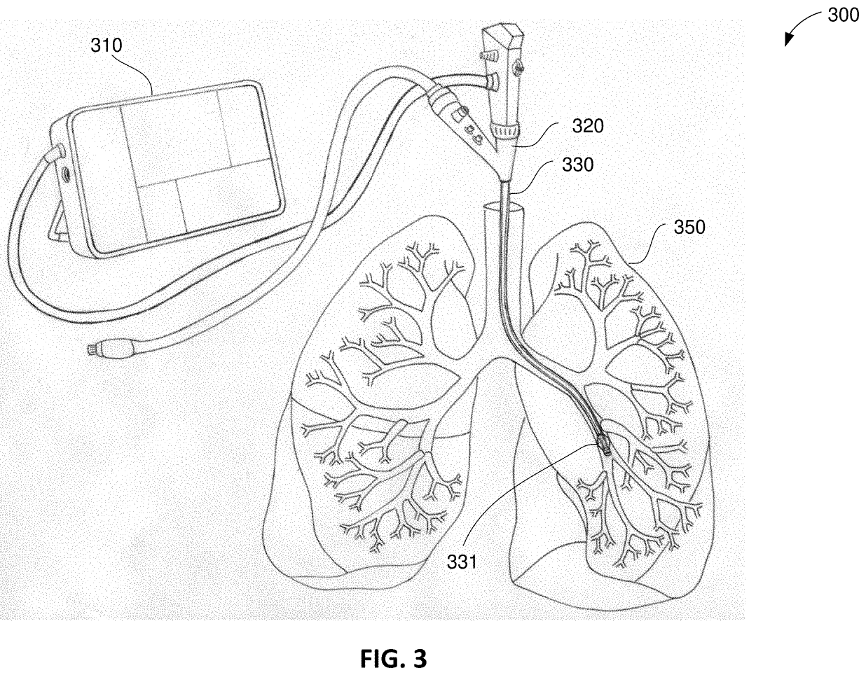

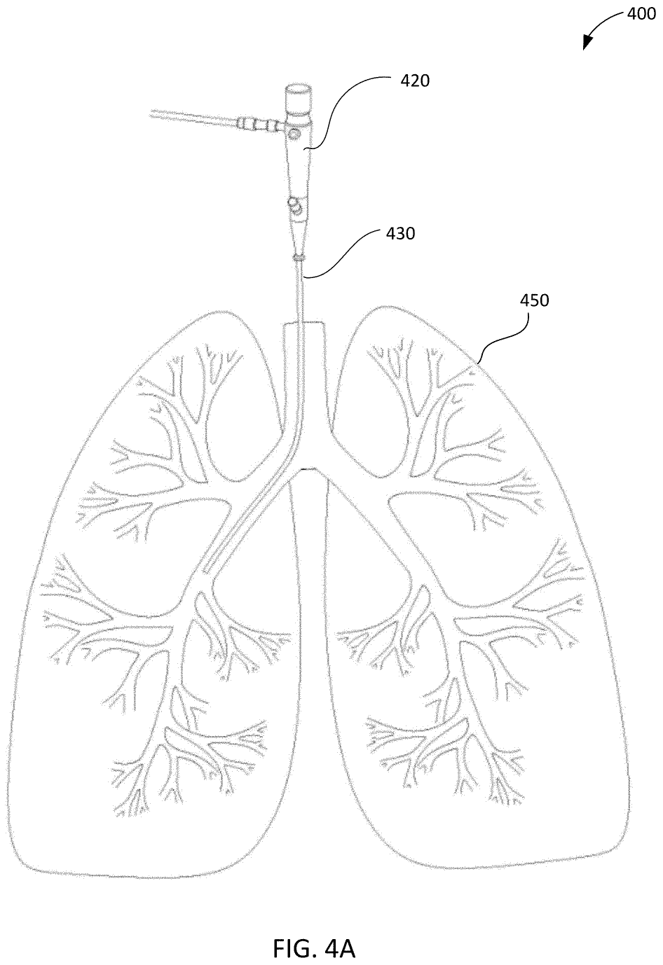

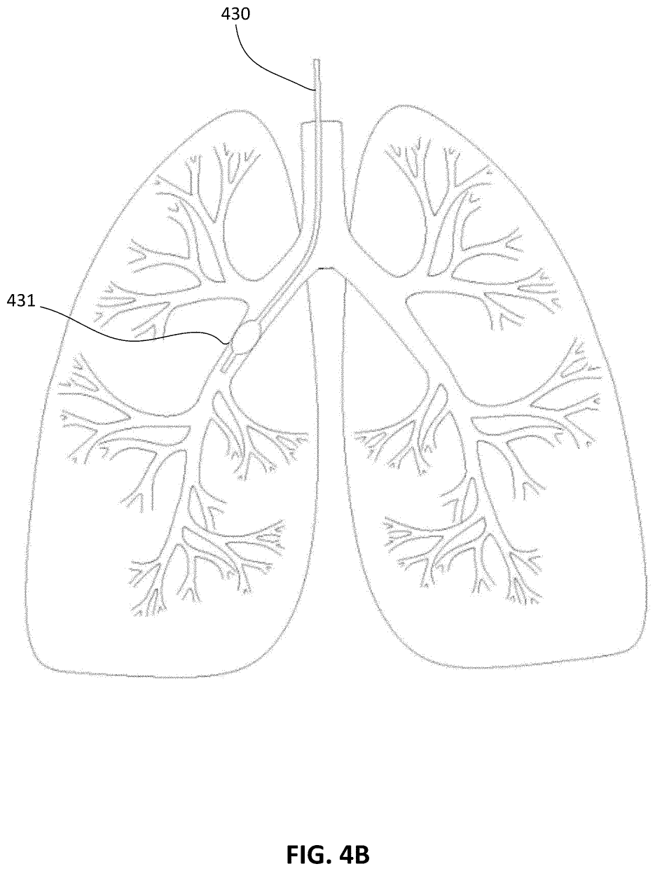

FIGS. 4A and 4B illustrate the use of a pulmonary isolation and ventilation apparatus 400 being within a patient's lung 450. In FIG. 4A the pulmonary isolation and ventilation apparatus 400 is shown initially operating in a first (visualization) mode in which the inner member is nested within the outer member and may be steered together to the target region of the lung, as shown. More particularly, a combination handpiece 420 is used to guide a multi-function endobronchial tube 430 into a lung 450. The combination handpiece 420 may be another example of the combination handpiece described above. The outer member (e.g., multi-function endobronchial tube 430) and/or inner may be steered by one or more controls on the handle. In the first mode, the user may guide the multi-function endobronchial tube 430 and inner member including visualization components into a particular target location of a lung 450. The location of the distal end of the multi-function endobronchial tube 430 may be verified visually through a display provided by a controller (not shown). Advantageously, the location of the outer member 430 may be verified without the need for x-ray or other imaging equipment. FIG. 4B shows an inflatable occlusion member 431 that may be used to anchor the multi-function endobronchial tube 430 and also isolate a region of the pulmonary system distal of the inflatable occlusion member 431.

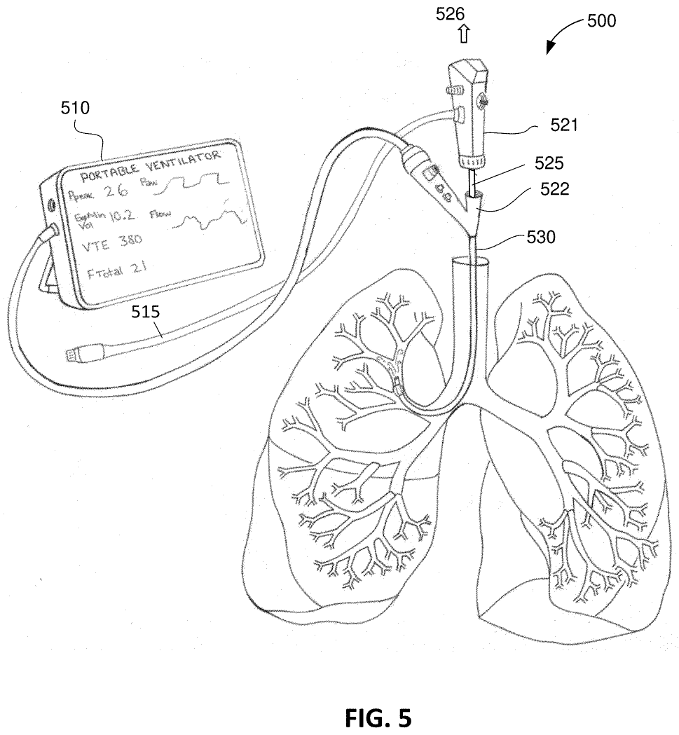

FIG. 5 shows a simplified diagram of a pulmonary isolation and ventilation apparatus 500 transitioning from operating in a first (positioning and/or visualization) mode into the second (ventilation) mode. The pulmonary isolation and ventilation apparatus 500 may include a controller 510, a first handle component (e.g., visualization handle) 521 coupled to the inner member 525, a second handle component (e.g., ventilation handle) 522 coupled to the outer member (multi-function endobronchial tube) 530. In this example, the distal end of the outer member has been positioned within the lungs at a target region and the anchor (balloon) inflated to isolate the sub-region of the lungs. The inner member, which may include visualization components such as one or more cameras, lights, etc., and/or one or more channels may then be removed by detaching the first handle portion 521 from the second handle portion 522 (e.g., by unscrewing, or otherwise detaching, and the first handle portion coupled to the inner member 525 may be withdrawn 526, as shown.

The controller may regulate the withdrawal by applying positive pressure through the inner member and/or the outer member to prevent a change in pressure at the distal end of the apparatus (e.g., within the lungs). Although FIG. 5 shows the first cable assembly 515 uncoupled from the controller, in practice the first cable may remain coupled to the controller or otherwise in communication with the controller, which may control the application of pressure through the inner member during withdrawal and/or insertion of the inner member from the outer member. A pressure sensor on the outer member (and/or inner member) may provide pressure input to the controller to regulate the pressure.

When operating in the second mode, the pulmonary isolation and ventilation system 500 may operate as a portable ventilator. The visualization handle 521 may be separated from the ventilation handle 522, as shown. Not shown is an elongate tube that contains camera and lighting components (for example, one or more fiber optic assemblies to provide light) that may be coupled to the visualization handle 521. Thus, as the visualization handle 521 is separated and removed from the ventilation handle 522, the elongate tube is withdrawn from the multi-function endobronchial tube 530. In some variations, the elongate tube is withdrawn from a channel. Thus, removal of the elongate tube may open up a lumen or channel within the multi-function endobronchial tube 530 that may be used to deliver gases and/or medicines to the lungs.

When coupled together, the visualization handle 521 and the ventilation handle 522 may form a combination handpiece similar to the combination handpiece 120 of FIG. 1A. The ventilation handle 522 may include a valve that can provide an air-tight seal for the opening where the visualization handle 521 attaches to the ventilation handle 522. Alternatively, in some examples the inner member 525 may be open or passively connected to atmosphere when inserting/withdrawing (in some examples the inner member may be vented.

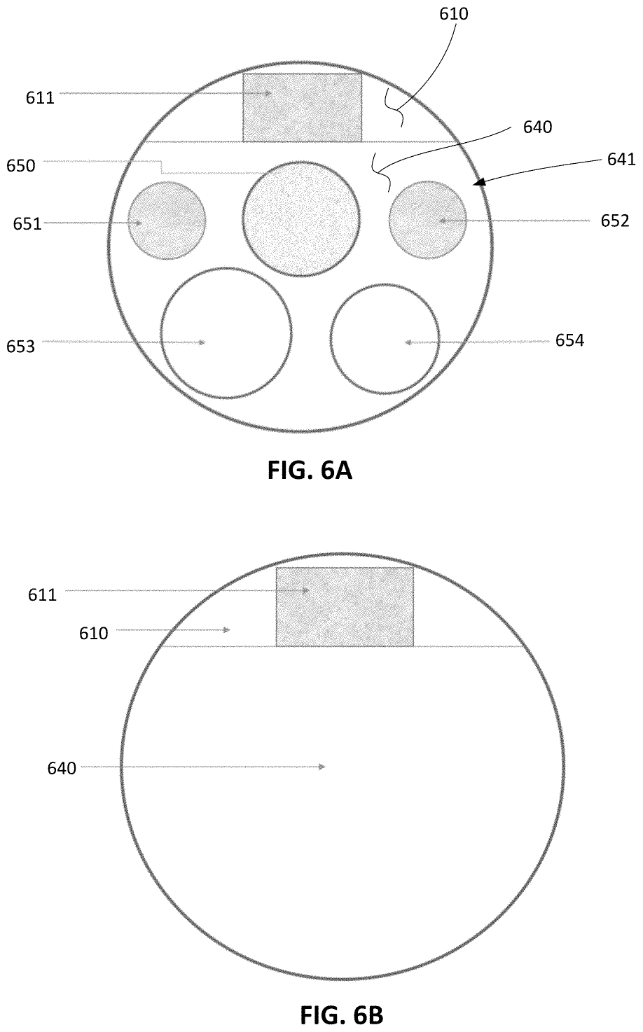

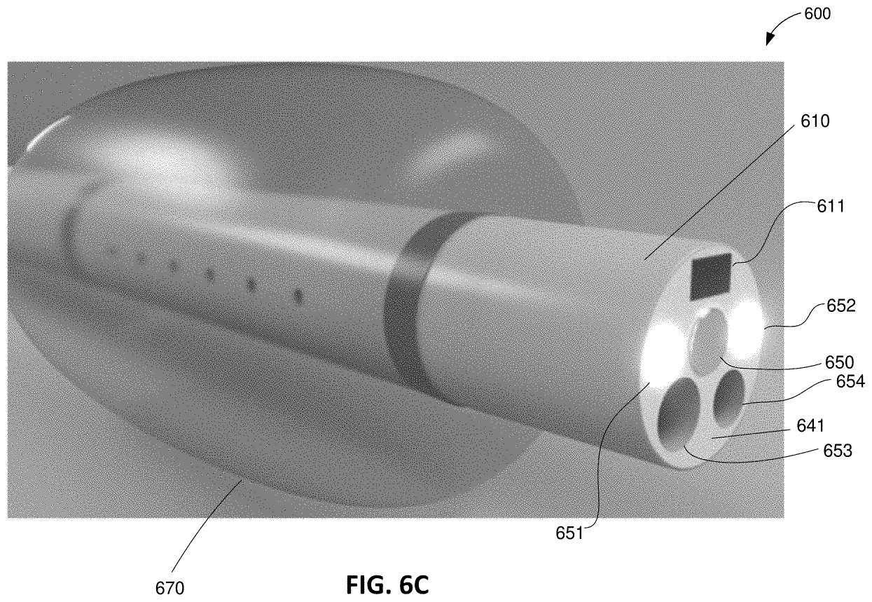

FIG. 6A shows an end view of an example on an outer member (multi-function endobronchial tube) 610 within which an inner member 641 is inserted and engaged. The multi-function endobronchial tube may include a first channel (lumen or bore 640). The outer member may also include a pressure sensor 611, which may also be a channel or lumen and/or may be within a channel or lumen. The pressure sensor 611 may provide the user pressure readings that may be present at the end of the multi-function endobronchial tube. Knowledge of the pressure may be critical to the application or delivery of gasses and medicines and/or the insertion and withdrawal of the inner member 641. For example, knowing the pressure may help prevent damage to the lungs when ventilating the patient. In some cases, a controller may provide air through the outer member 610 and/or the inner member 641 based at in part on determined air pressure measurements from the pressure sensor 611.

The large bore 640 of the outer member 610 in FIG. 6A houses the inner member (elongate tube) 641 which, in turn, may include a camera 650, a first light source 651, a second light source 652, a suction channel 653, and an irrigation channel 654. The elements shown here as part of the inner member 641 are exemplary and are not meant to be limiting. Thus, in some variations the outer member 610 and/or the elongate tube 641 may include other light sources, more or fewer additional channels, and so forth.

The camera 650 may be used to provide endoscopic images to the user. In particular, the camera 650 may enable the user to locate and identify bronchial treatment areas. The first light source 651 and the second light source 652 may be used to provide illumination to the distal end of the apparatus. In this manner, sufficient light may be delivered to the bronchial treatment areas to enable the camera 650 to capture detailed images. The pressure (positive or negative) channel 653 may provide positive or negative pressure to the distal end of the apparatus. The irrigation channel 654 may provide saline or any other feasible liquid that may be used to irrigate a region near the distal end of the apparatus.

The inner member may engage with the outer member in sealing manner, e.g., so that there is little or no air gap between the two when the inner member is inserted into the outer member. In some examples one or more seals (e.g. 0-rings) may be included along the length of the elongate bore and/or handle(s). Alternatively in some examples the space between the inner and outer members may be vented. The large bore 640 may be lubricated (e.g. by a lubricious material). The large bore 640 may have an inner region that is keyed to the outer surface of the inner member.

The elements within the inner member (elongate tube) 641 may be coupled to a first handle portion (e.g., visualization handle) as described above. In this manner, when the visualization handle is separated from the ventilation handle, the inner member 641 may be removed from the second channel 640 and therefore from the outer member (multi-function endobronchial tube) 610. The empty second channel 640, which was previously occupied by the inner member 641 may now be used to provide gases or therapeutic agents to the bronchial treatment area.

FIG. 6B shows the end view of an example of an outer member (e.g., multi-function endobronchial tube) 610 after the inner member (elongate tube) 641 has been removed from the large bore 640. The pressure sensor 611 remains in the outer member 610. In this manner, pressures at the end of the multi-function endobronchial tube 600 may be monitored and controlled. The empty second channel 640 may be used to deliver gases and/or therapeutic agents to the patient.

FIG. 6C shows a perspective view of the distal end of the apparatus 600 including an outer member 640 with a pressure sensor 611, and a removable inner member 641 including the camera 650, the first light source 651, the second light source 652, the positive/negative pressure channel 653 and the irrigation channel 654. An inflatable occlusion member 670 is shown in an inflated state.

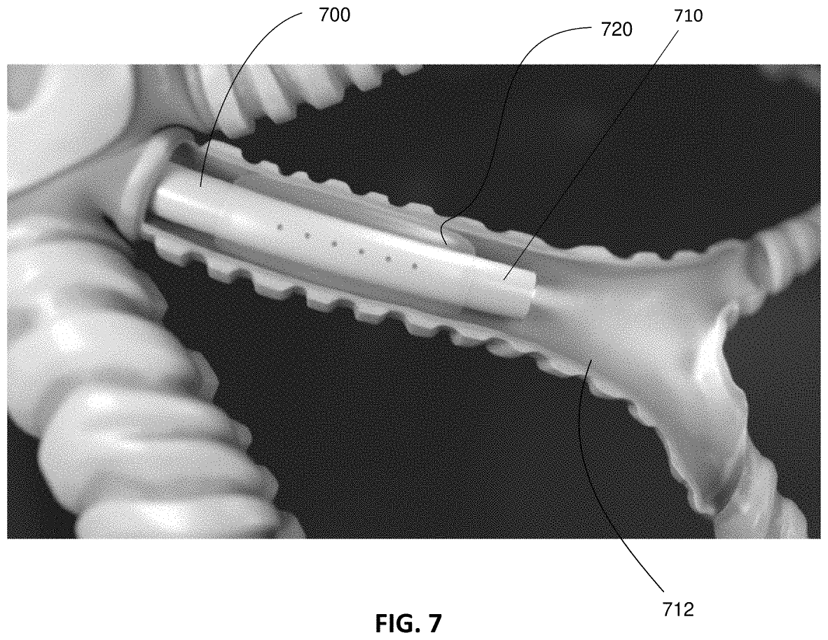

FIG. 7 shows a distal end of an apparatus 700 in situ. In this example, the apparatus 700 (including an outer member 710 surrounding an inner member) has been guided into position in a bronchial tube 712. Additionally, an inflatable occlusion member 720 is shown inflated to both anchor the outer member 710 in place as well as isolate a portion of the bronchial tube 710. The isolation may enable pulmonary treatment to be restricted to a particular region of the lungs.

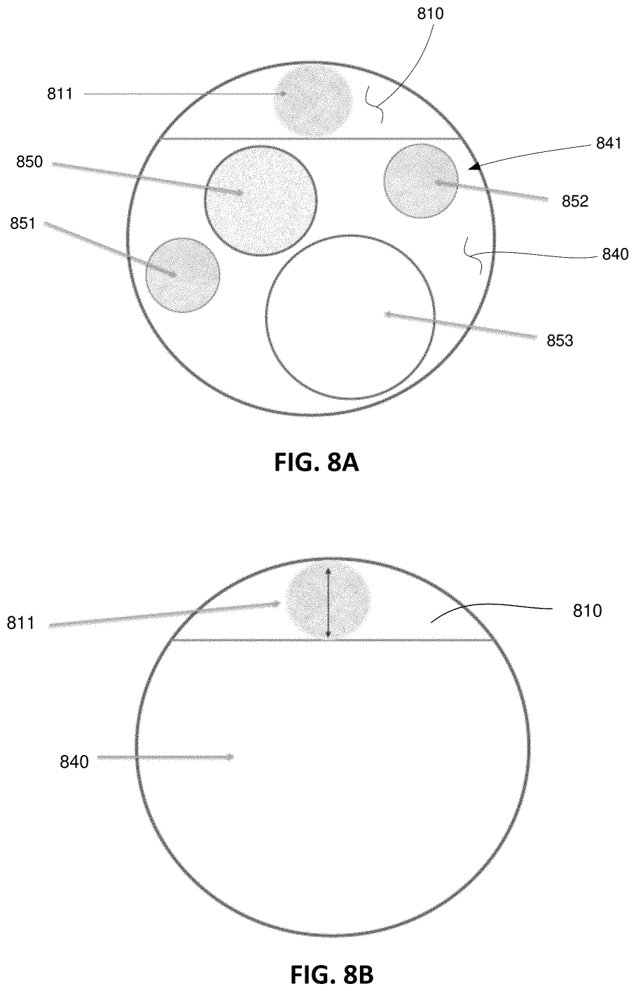

FIG. 8A shows an end view of an example of an apparatus including an outer member 810 and an inner member 841. The outer member (multi-function endobronchial tube) 810 may include a large lumen, channel, or bore 840 in which the inner member may engage. The first outer member 810 may include pressure sensor 811, which may include a lumen (pressure sensing lumen).

In FIG. 8A the large bore of the outer member includes an inner member (elongate tube) 841 that includes a camera 850, a first light source 851, a second light source 852, and a multi-function channel 853. In some embodiments, the multi-function channel 853 may provide suction through negative pressure or may provide saline or any other liquid to irrigate an area or may provide positive pressure. The inner member 841 may be removed from the multi-function endobronchial tube 800 leaving the second channel 840 empty.

FIG. 8B shows the end view of the outer member after the inner member 841 has been removed from the large bore 840. The pressure sensor 811 remains with the outer member 810. In this manner, pressures at the end of the apparatus may be monitored and controlled. The empty large bore 840 may be used to deliver gases and/or therapeutic agents to the patient.

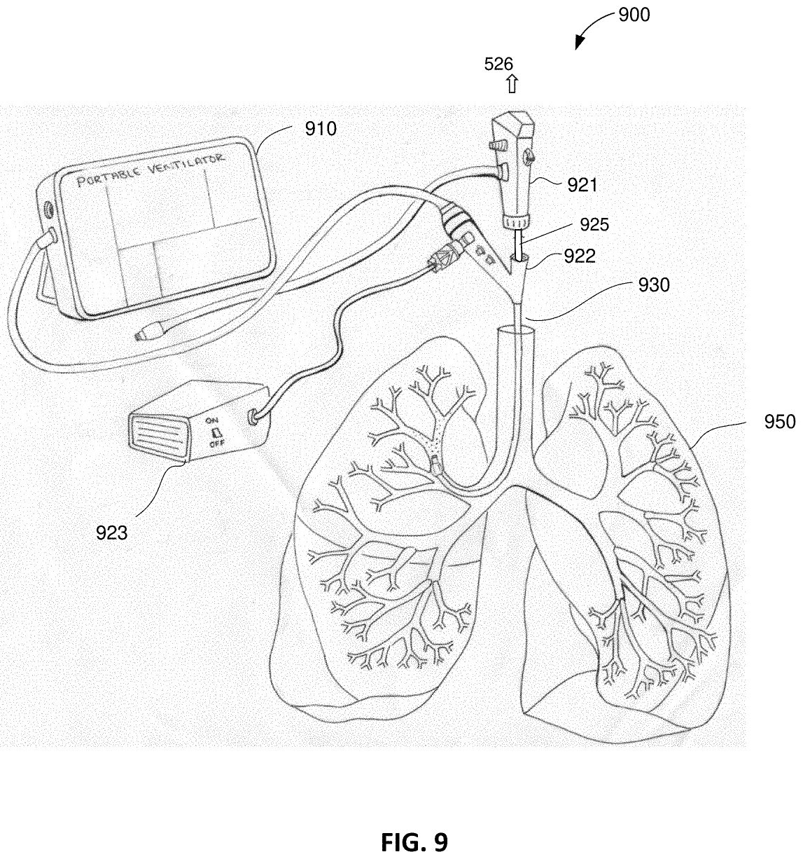

FIG. 9 shows an example of an apparatus 900 for visualization and ventilation of a bronchial region transitioning from the first (e.g., navigation/visualization) mode to the second (ventilation) mode. The pulmonary isolation and ventilation apparatus 900 may include a controller 910, a visualization handle 921 coupled to an inner member 925, a ventilation handle 922 coupled to an outer member 930, and a nebulizer 923.

In the ventilation mode, the outer member 930 may be inserted into a treatment position in a patient's lungs 950. The visualization handle 921 may be separated from the ventilation handle 922 and the controller 910 may control ventilation (administration of gases) to the patient's lungs 950. The nebulizer 923 may also be coupled to the ventilation handle 922. The controller may also couple to (not shown) the nebulizer and may control the application of material from the nebulizer. The nebulizer 923 may deliver any gas, liquid or atomized liquid therapeutic agents through the ventilation handle 922 and out of the apparatus in the target lung region.

In any of the apparatuses and methods described herein an agent, including but not limited to a nebulized agent, may be applied through the apparatus, including through the large bore of the outer member. For example, a nebulizer such as that shown in FIG. 9 may be used to generate particles (aerosol particles, etc.) for deliver through the apparatus, including through a ventilation circuit of the apparatus. As shown in FIG. 9, the nebulizer may be coupled to one or both portion of the handpiece for delivery through a lumen (e.g., the large bore lumen of the outer member) and into the target region of the lungs. In FIG. 9 the nebulizer is coupled to a handpiece or to tube of ventilator; in some examples the nebulizer may be coupled to a Y-junction coupled to the tube and therefore to a lumen of the apparatus. In any of the apparatuses described herein the tubing to/from the handpiece and/or the ventilation circuit may include separate and/or branched tubing (e.g., for inspiration/exhalation).

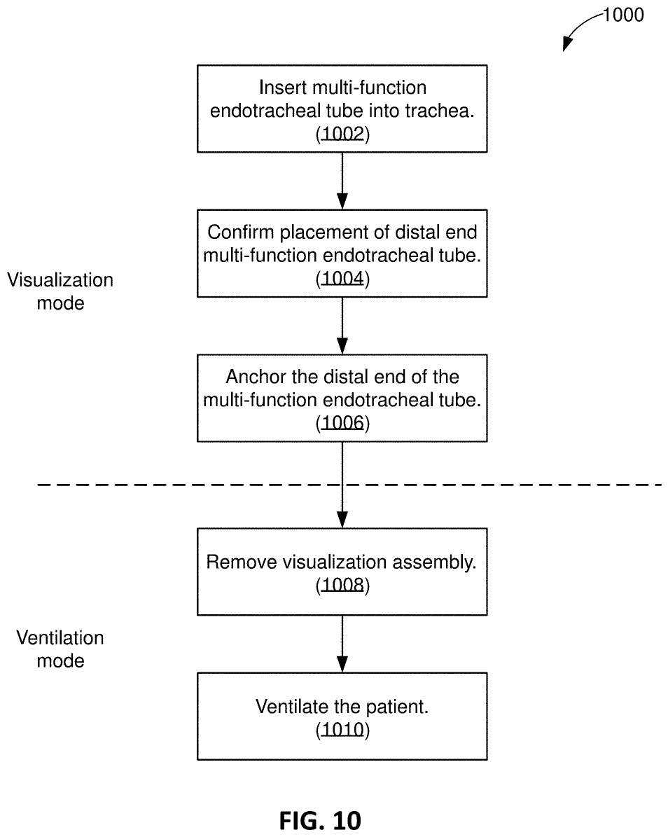

FIG. 10 schematically illustrates an example of a method 1000 for operating a pulmonary isolation and ventilation system. Some examples may perform the operations described herein with additional operations, fewer operations, operations in a different order, operations in parallel, and some operations differently. The method 1000 is described below with respect to the pulmonary isolation and ventilation apparatus such as that shown above, however, the method 1000 may be performed by any other suitable systems or devices similar to those described herein.

A pulmonary isolation and ventilation apparatus may be inserted while operating in a visualization mode 1002. For example, an outer member (multi-function endobronchial tube) may be inserted into a patient's trachea. The apparatus may be inserted with the inner member engaged within the lumen of the outer member. The inner member may be engaged so that steering the outer (or inner) member also steers the inner (or outer) member. In some examples the user may guide or steer the outer member using a handpiece which may operate one or more guidewires; alternatively or additionally the inner member may be steered. Next, the placement of the distal end of the apparatus (including the outer member) may be confirmed. For example, a camera (which may be part of the inner member) may capture images that may be displayed on the controller. Using the images, the user can confirm that placement of the distal end of the outer member is correct.

Next, the distal end of the multi-function endobronchial tube may be anchored. For example, an inflatable occlusion element disposed proximate to the distal end of the multi-function endobronchial tube may be inflated, causing the inflatable occlusion element to contact an inner wall of a lumen and thereby anchor the multi-function endobronchial tube.

Once the position is confirmed, the region may be treated through the inner member (e.g., through one or more channels of the inner member) and/or the inner member may be removed leaving the large bore of the outer member in fluid communication with the isolated region of the lungs. For example, the apparatus may be transitioned from the visualization mode to a ventilation mode 1008 by removing the inner member, including the visualization assembly. For example, a visualization assembly may include the visualization handle attached to the inner member (elongate tube) that includes one or more elements associated with capturing images from the distal end of the apparatus outer member. Removal of the inner member may provide a channel or lumen (large bore) that may be used to ventilate the patient without disturbing the positioning of the anchor and the outer member. Thus, removal of the inner member could be, at least in part, a removal of the visualization assembly. In some versions, the visualization handle may be separated from the ventilation handle to remove the visualization assembly and the controller may regulated the pressure within the distal end region (and within the target lung region) by applying pressure as the device is removed to prevent a vacuum forming.

The pulmonary isolation and ventilation apparatus may then ventilate the patient through the large bore 1010. For example, the controller may provide air through the ventilation handle and the large bore of the outer member. In some variations, the inflatable occlusion member (anchor) may isolate a region of the lungs to create a treatment region that may be a subset of a complete lung. In some cases, the treatment region may be limited to a lobe or a portion of a lobe of a lung. In some other examples, the controller may ventilate the patient in accordance with pressure readings from a pressure sensor included in the outer member.

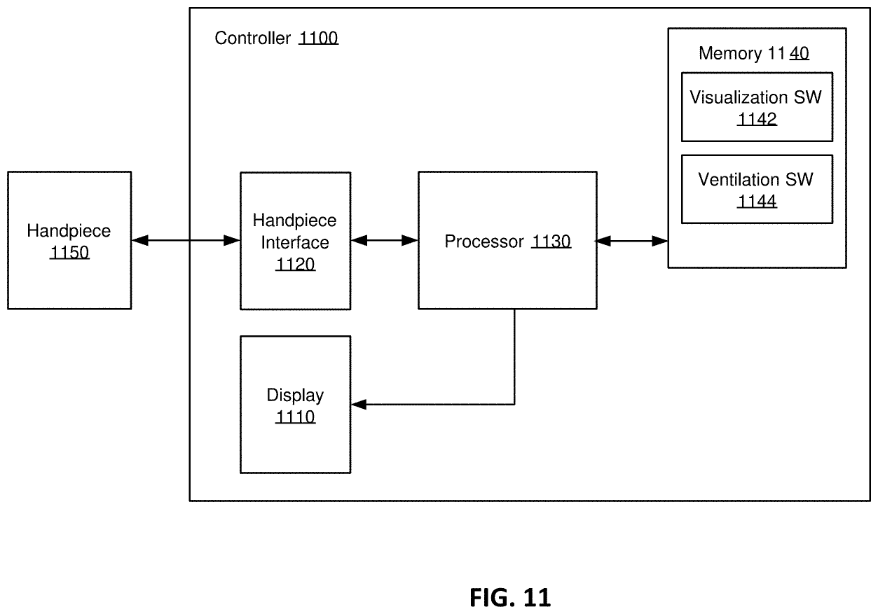

FIG. 11 shows a block diagram of an example of a controller 1100 that may be one example of the controller of any of the apparatuses described herein. Persons having skill in the art will recognize that the controller 1100 may be examples of the controller 310 of FIG. 3, the controller 510 of FIG. 5, the controller 910 of FIG. 9, or any other feasible device. The controller 1100 may include a display 1110, a handpiece interface 1120, a processor 1130, and a memory 1140.

The handpiece interface 1120, which is coupled to the processor 1130, may be used to interface with any feasible handpiece, such as handpiece 1150. The handpiece 1150 may be an example of the handpiece 120 of FIG. 1, or any other feasible handpiece. The handpiece 1150 may be coupled to a multi-functional endobronchial tube (not shown). The multi-functional endobronchial tube may include visualization components (fiber optic lighting elements, one or more cameras, etc.) and ventilation components (lumens or channels to provide air to selected regions of the lungs). Additionally, the multi-functional endobronchial tube may include a pressure sensor disposed proximate to its distal end.

The display 1110, which is also coupled to the processor 1130, may be used to display, interface, and control any feasible handpiece, such as the handpiece 1150. For example, the display may show images from a camera, and provide an interface to receive input from a user to direct visualization and/or ventilation operations.

The processor 1130, which is also coupled to the memory 1140, may be any one or more suitable processors capable of executing scripts or instructions of one or more software programs stored in the controller 1100 (such as within memory 1140).

The memory 1140 also include a non-transitory computer-readable storage medium (e.g., one or more nonvolatile memory elements, such as EPROM, EEPROM, Flash memory, a hard drive, etc.) that may store the following software modules: a visualization software (SW) module 1144 to process image data received by the handpiece interface 1120; and a ventilation SW module 1146 to control ventilation operations provided through the handpiece 1150. Each software module includes program instructions that, when executed by the processor 1130, may cause the controller 1100 to perform the corresponding function(s). Thus, the non-transitory computer-readable storage medium of memory 1140 may include instructions for performing all or a portion of the operations described herein.

The processor 1130 may execute the visualization SW module 1144 to control visualization operations performed by the controller 1100. For example, execution of the visualization SW module 1144 may cause image data captured by a camera within the multi-function endobronchial tube to be displayed on the display 1110. In some variations, execution of the visualization SW module 1144 may enable the user to control how much light is provided by light sources toward a distal end of the multi-function endobronchial tube.

The processor 1130 may execute the ventilation SW module 1146 to control ventilation operations delivered via the handpiece 1150 to a patient. For example, execution of the ventilation SW module 1146 may cause the processor 1130 to determine a pressure near a distal end of the multi-function endobronchial tube from a pressure sensor. Further execution of the ventilation SW module 1146 may cause the controller 1100 to provide air to the patient in accordance with the determined pressure, thereby preventing damage to pressure sensitive lung tissue.

### Localized Drug Delivery

In general, the methods and apparatuses described herein may be used to deliver targeted and localized drug delivery, and in particular, delivery of a nebulized therapeutic (e.g., drug) within a predetermined region of the lungs. For example, the methods an apparatuses described herein may deliver specific and targeted nebulized drug therapy. Nebulized therapeutic agents are often delivered via inhalation through the nose and/or mouth. However, it has long been known that much of the nebulized agent is deposited (and often absorbed) within the nasal passages, mouth, throat, and esophagus before even reaching the lungs. Often this is undesirable and may result in lower drug levels in the lungs or other targeted regions.

In contrast, the methods described herein may be used to isolate and deliver agents, including but not limited to nebulized agents (including drug agents, surfactants, etc.) within targeted subregions of the lungs that may be isolated as described herein. This may allow for higher and more precisely controlled levels of the agent(s) with in the targeted sub-region. For example, an apparatus as described herein may be guided using the combined outer member and inner member to a target bronchial region and may isolate the region by expanding the anchor (e.g., balloon, mesh, etc.) to secure the distal end of the apparatus in communication with the target region. The inner member may be removed as described herein, and an agent (e.g., nebulized agent) may be applied through the large bore of the outer member. Alternatively, the agent may be applied through one or more channels of the inner member while it remains in place. Before, during or after the delivery of the agent, ventilation may be applied through the apparatus as described herein.

In general, the large bore of the outer member (and/or the channels of the inner member) may be configured, including coated, treated, layered, etc. to resist retaining the agent, such as the nebulized agent. For example, the surface of the large bore of the outer member may comprise (including being formed of, coated with, or otherwise include) hydrophobic material or the like. In some examples the surface may be charged to resist contact with the charged agent (or charged particles/droplets including the agent).

In some examples an agent may include a surfactant to be applied to the lungs or a region of the lungs. For example the apparatus described herein may be used to treat a pediatric patient to deliver a surfactant within the lungs or a region of the lungs. In some examples the apparatus may deliver an agent that may include an antibiotic to be applied to the lungs or a region of the lungs. For example the apparatus described herein may be used to treat a patient with lobar pneumonia to deliver an antibiotic within the affected regions of the lungs. FIG. 18 illustrates an example of a representation of a patient having right lower lobar pneumonia in which an apparatus as described herein is inserted into the region affected by pneumonia (e.g., the segments affected by the infection). The distal end region 1805 of the apparatus may be navigated to the affected region, and sealed to the region using a sealing anchor as described above. The outer member (e.g., sleeve) may then be used to add or remove material from this segment of the lungs, including adding an agent (e.g., localized treatment using an active agent).

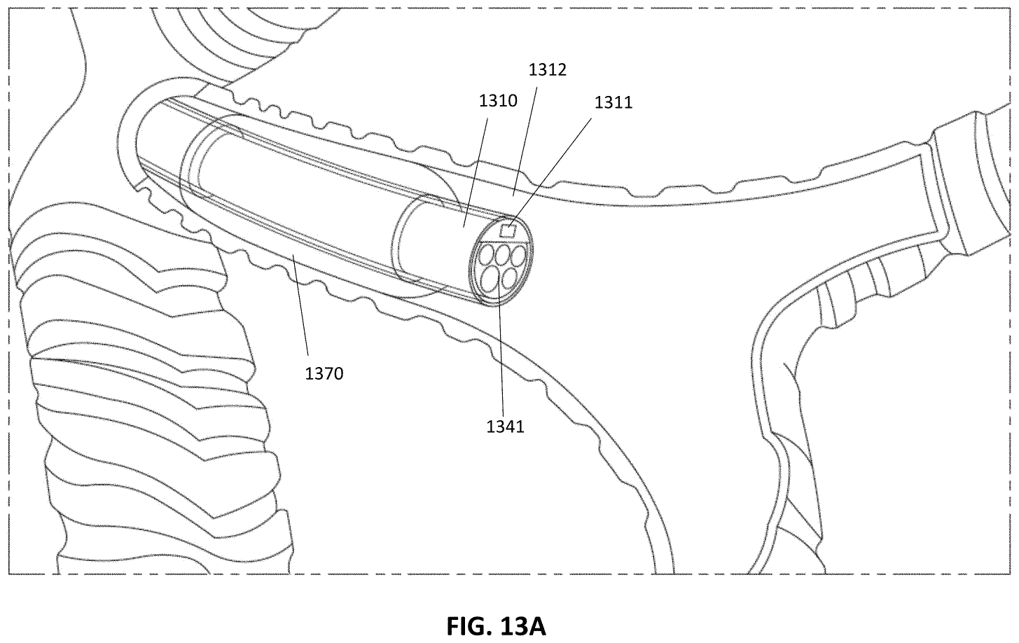

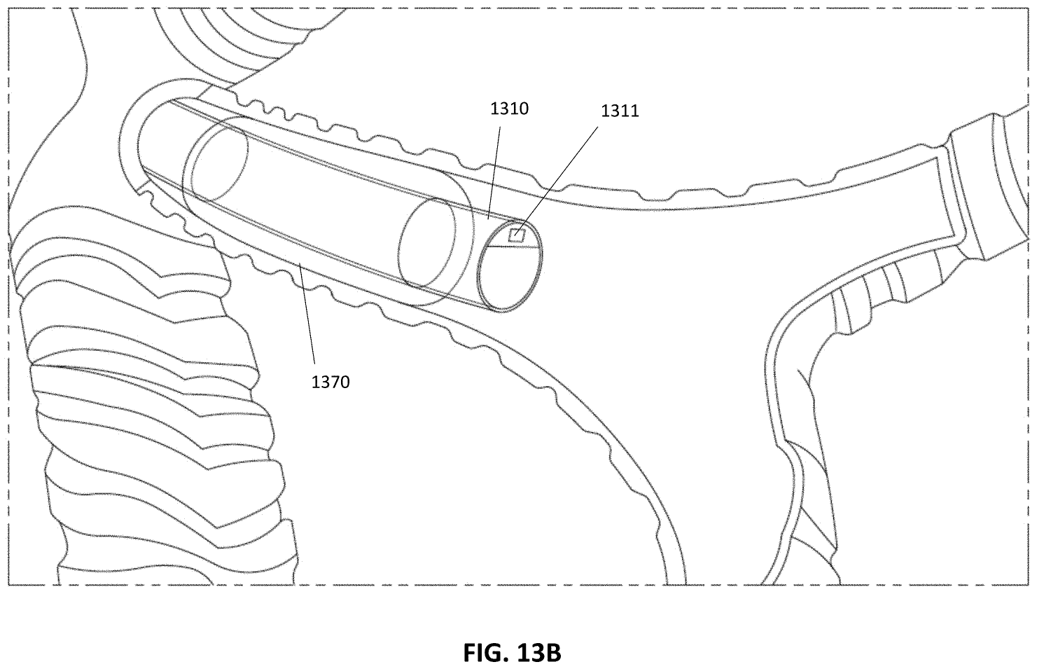

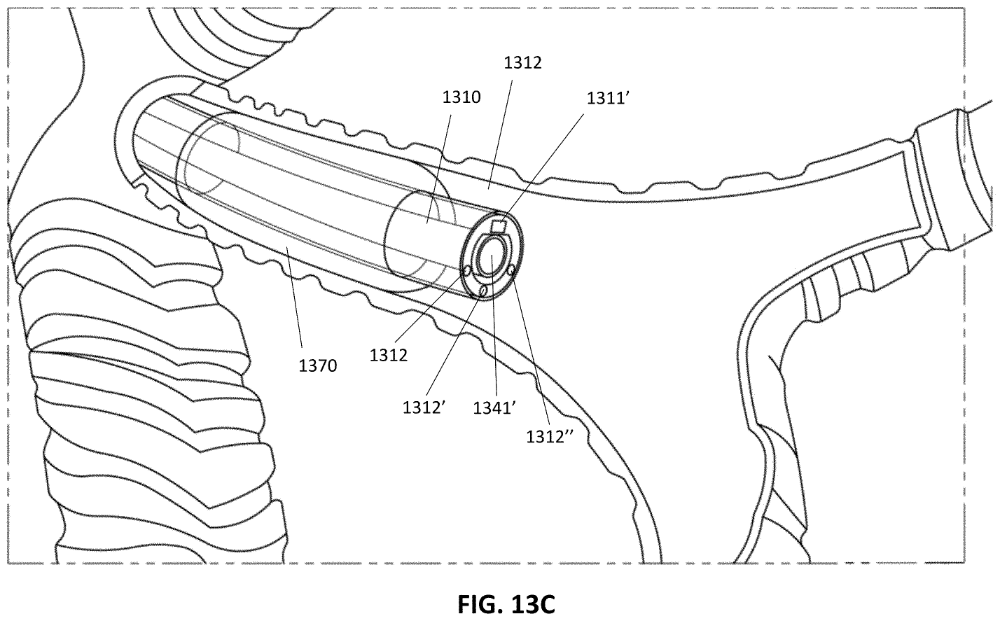

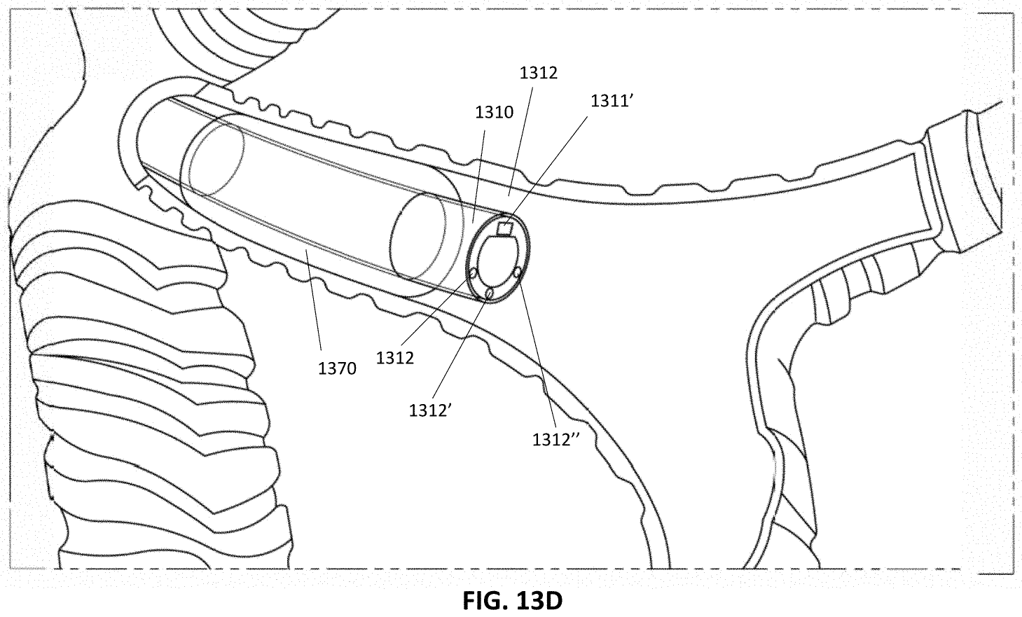

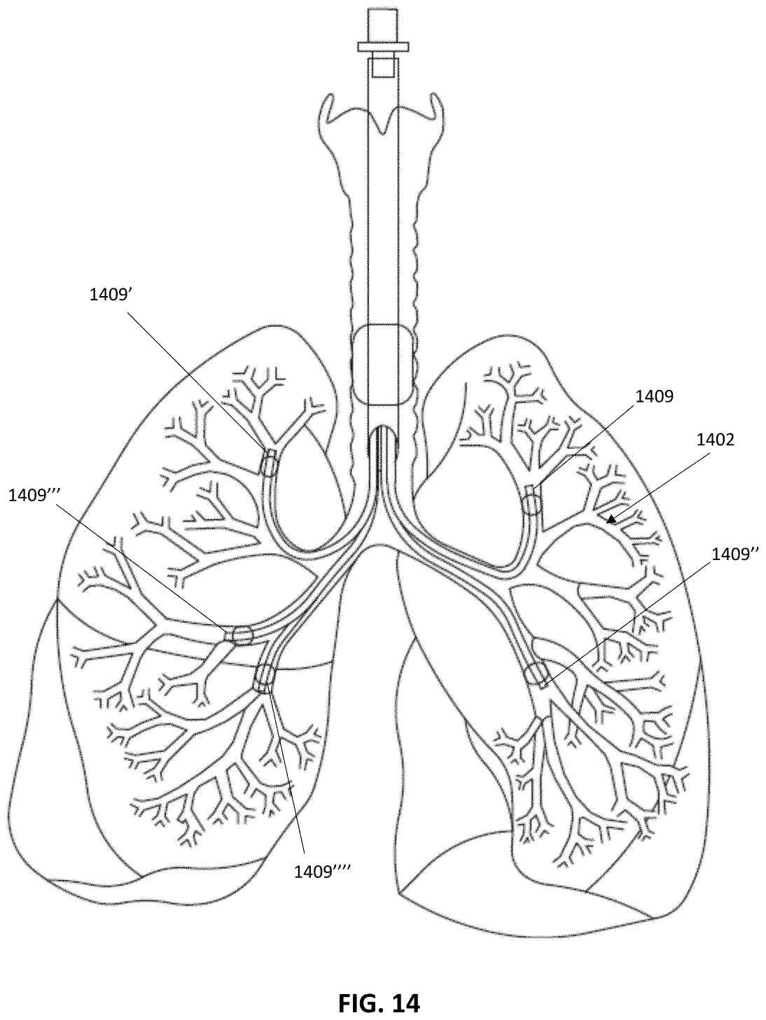

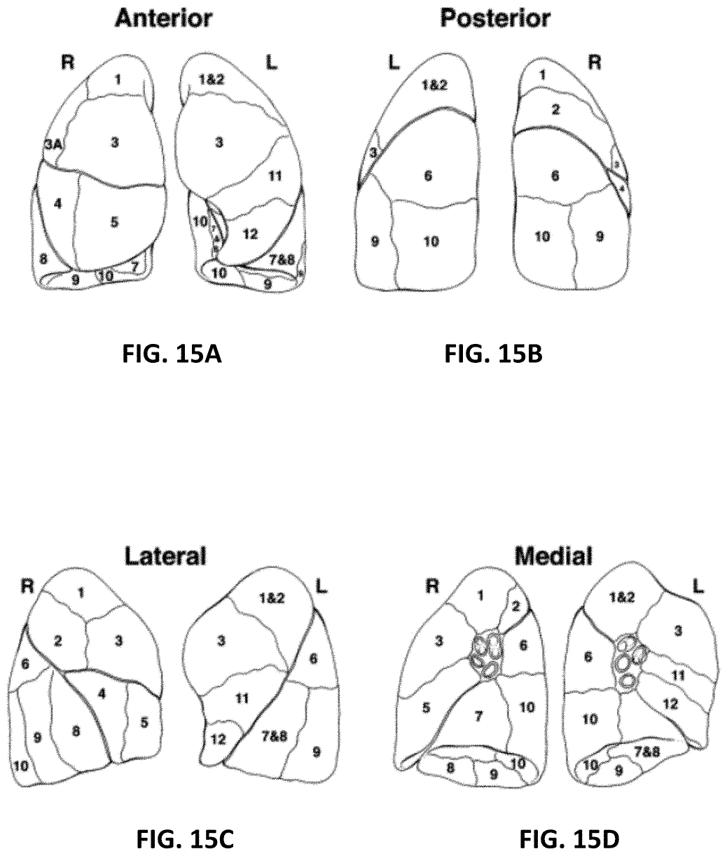

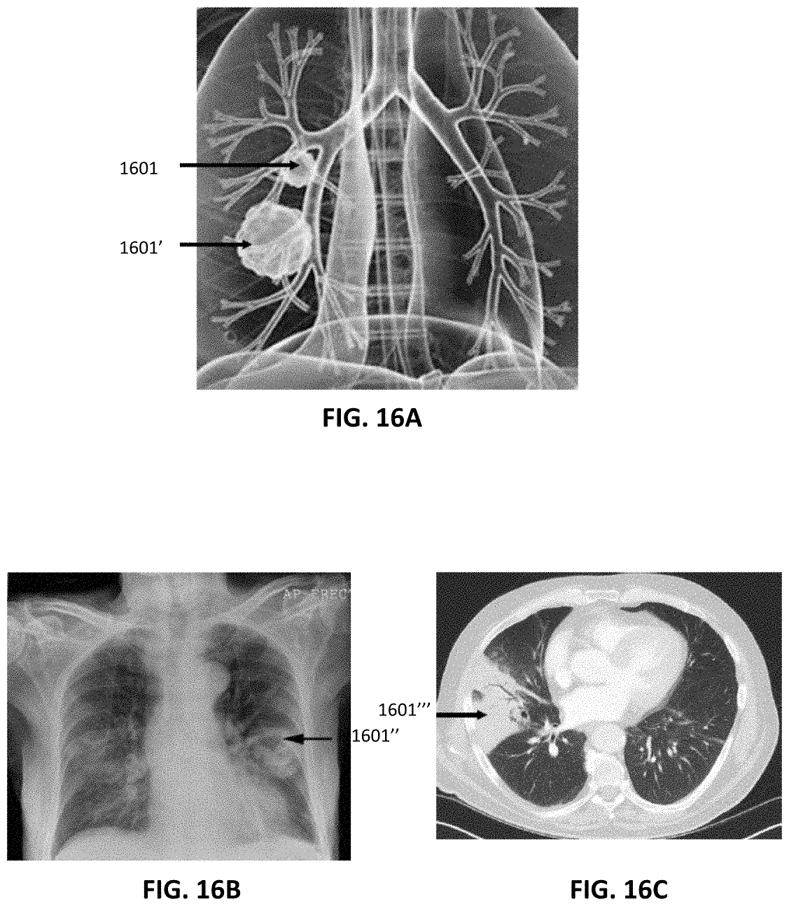

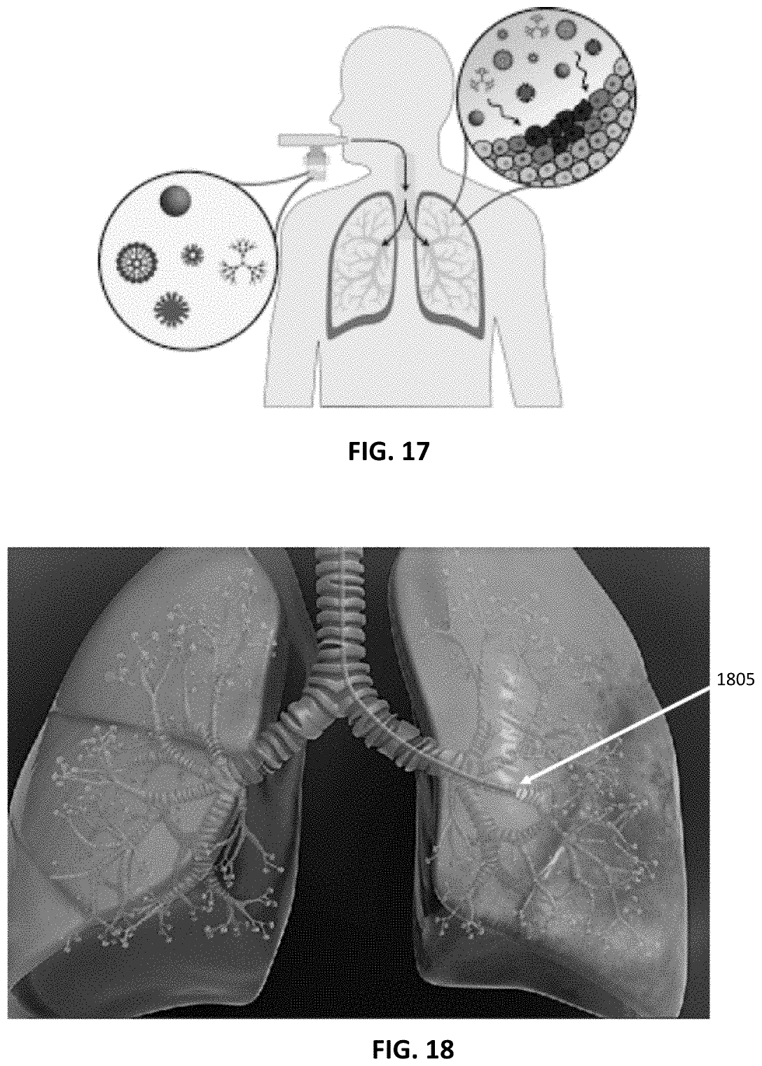

For example, in FIG. 18, an outer member configured as a sheath may be used with a commercially available inner member (e.g., endoscope, such as a bronchoscope). For example, the system shown in FIGS. 13A-13B or 13C-13D may be used. The distal end of the apparatus may be advanced within a bronchus and the sealing anchor may be expanded, as shown in FIGS. 13A-13B and 13C-13D. In this example the sealing anchor may be an annular balloon, though other sealing anchors could be used, including other balloon shapes. The apparatus may be positioned with the inner member (e.g., bronchoscope) inserted. Thus, the distal end has may be positioned within the region of the lung affected by pneumonia (bronchial region 1805) that is to be treated, and the anchor may be deployed to secure the distal end region of the apparatus within the bronchial region as shown, and to seal it within the bronchial region.

The inner member, which in some examples may include a camera, a pair of lights and two working channels, may be withdrawn from the large bore lumen of the outer member (sleeve). The outer member (e.g., sleeve) may include a pressure sensor as described above. Alternatively, the outer member (e.g., sleeve) may include a number of sensors, light source, etc. and the inner member may be steerable but may not include sensors (or may include some sensors). Thus, the apparatus includes just the outer member and access to the region distal of the anchor is provided through the sleeve. The lumen of the outer member may therefore act as a working channel. The apparatus may otherwise be used as described above. Thus, once the internal endoscope (e.g., bronchoscope) is removed, what remains in place is a large working channel that allows for all kind of different therapeutic uses in selective areas of the lungs, including the application of one or more agents.

Any appropriate agent may be delivered, including a drug (small molecule drug, etc.), and antibodies, a surfactant, particles (e.g., microparticles, nanoparticles, etc.), vesicles (e.g., including cells, such as but not limited to stem cells), viral vector, extracted vesicles, etc. In some examples the methods and apparatuses described herein may be used for gene therapy, including gene therapy to treat a lung or region of lung, and/or a tumor within a lung or region of lung.

Selective positioning of the apparatus as described herein may allow for the safe ventilation and lung recruitment under continuous airway pressure monitoring, and/or for the local delivery of drugs, antibody, etc. (i.e., nebulization of therapies, including therapies for treating COVID-19)

The apparatuses described herein may be used in an emergency setting, or in any setting that would benefit from airway management. In particular, these apparatuses may be useful in setting in which difficult airway management may require the use of a ventilation applied to all or a region of the lungs. In some examples the apparatus may include or be configured as a disposable bronchoscope. In patients including an obstruction (e.g., congenital abnormality, acute obstruction, etc.) the apparatuses described herein may offer steerable visualization and placement of the tip, anchoring the tip, e.g., beyond the obstruction, and ventilation through the apparatus.

The apparatuses described herein may be used with any appropriate ventilation type of system. For example, in some examples the apparatuses described herein may be used with or as part of a jet ventilation apparatus, which may apply air continuously or intermittently at a desired flow rate.

### Lung Inflation

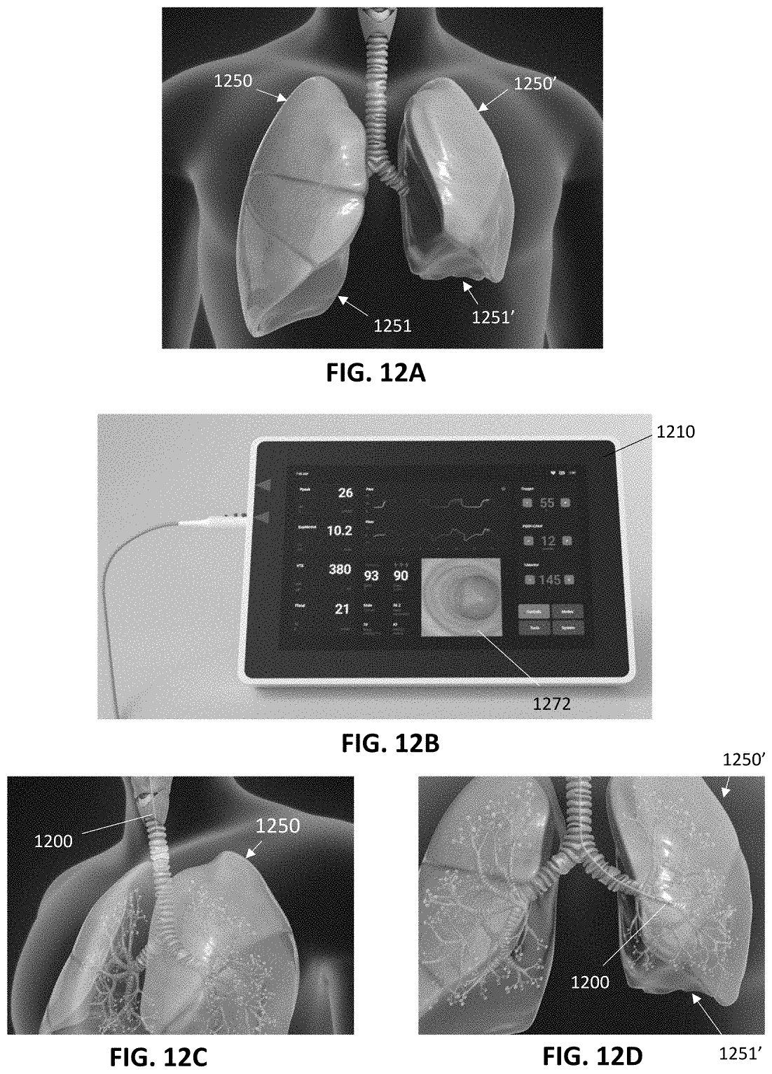

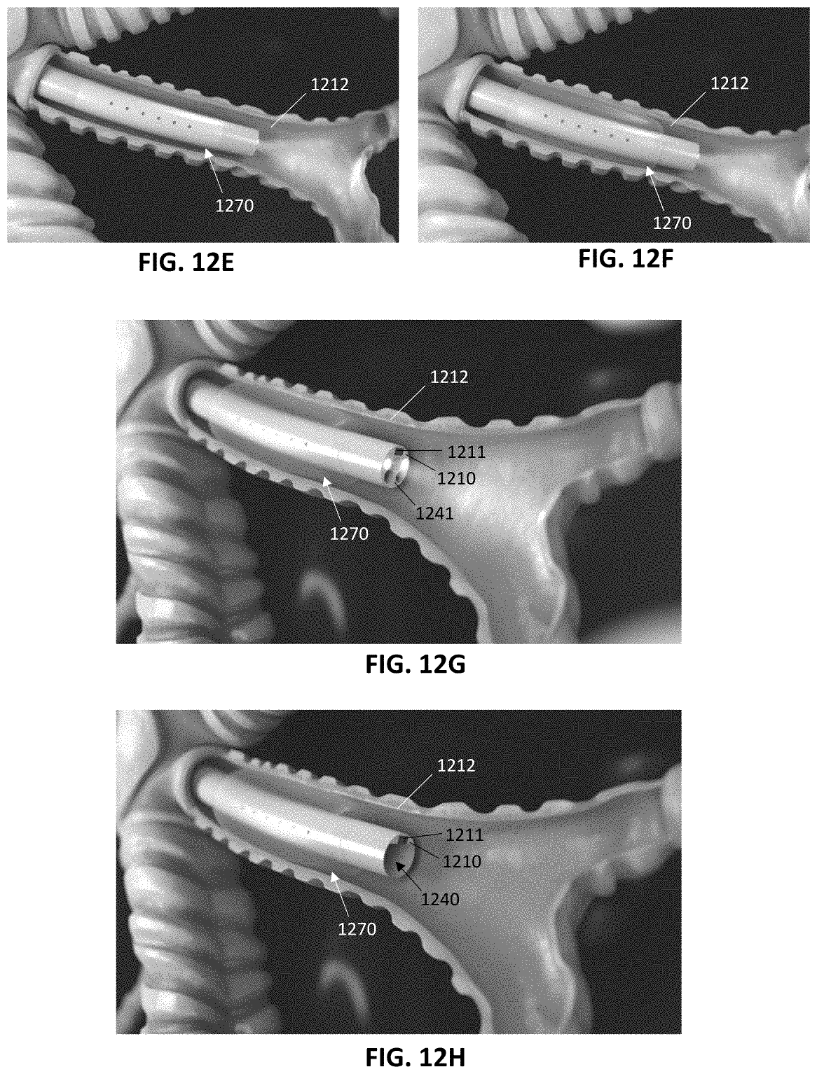

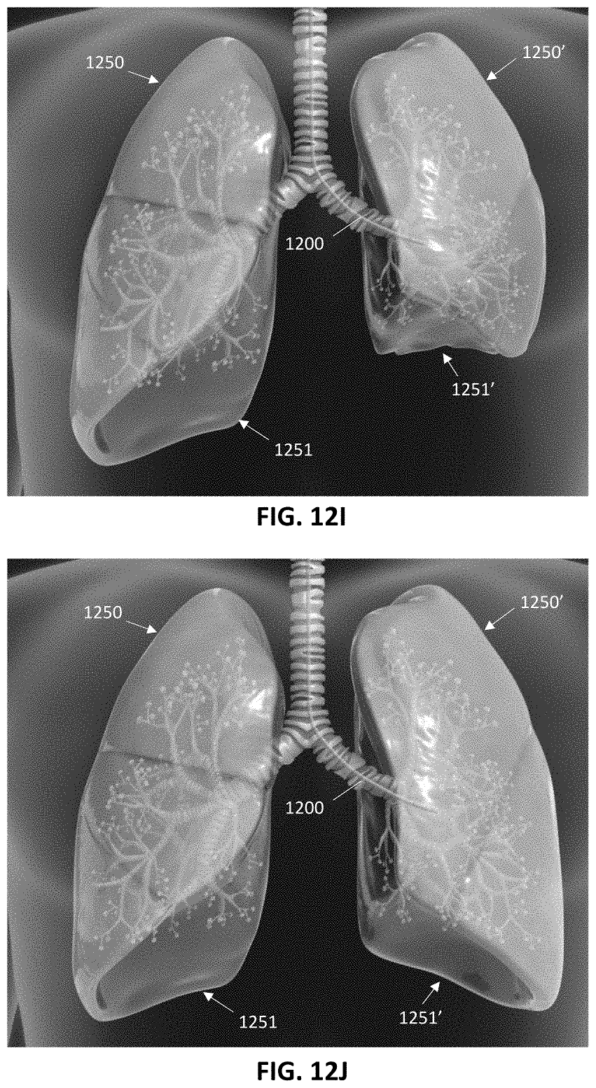

FIGS. 12A-12J illustrate one example of an apparatus and method of use as described herein. In this example the apparatus may be used to re-inflate a portion of the lungs that has collapsed. This may be useful in treating a living patient, or in treating an organ (e.g., lung) donor. For example, lungs that are donated but not deemed suitable for transplantation (e.g., because of collapse or partial collapse) may be treated as described herein to increase the PaO₂ level; once the PaO₂ has improved, the donated lungs can be accepted by the transplant center, thereby increasing the number of transplanted patients. Selective ventilation as described herein may allow for recruitment of areas that are otherwise atelectatic and not breathing.

For example, FIG. 12A shows an example of lung 1250′ that is partially collapsed 1251′ on the left side of the patient, as compared to the lung 1250 on the right side of the patient that is not collapsed 1251.

An apparatus as described herein may include an insertable outer member comprising a flexible endobronchial tube having a lumen extending therethrough and a pressure sensor at a distal end, an elongate inner member configured to sealingly fit within the lumen, a first handpiece portion coupled to the outer member, a second handpiece portion coupled to the inner member and configured to releasably engage the first handpiece portion; and a controller configured to couple to the first handpiece portion and to coordinate operation of the apparatus during use.

FIG. 12B shows an example of a prototype controller (not visible), including an input and an output (e.g., touchscreen display) 1210. The output 1272 in this example shows the camera image from the inner member after inserting the inner and outer member through the patient's esophagus into the lungs, as shown in FIGS. 12C and 12D. In this example, the apparatus 1200 is inserted through the branches of the branchia into a region that is near the collapsed portion 1251, where it may be anchored, a shown in FIGS. 12E-12H in more detail. In FIG. 12E the distal end of the apparatus is advanced within a bronchus 1212 with the anchor 1270 collapsed. In this example the anchor is an annular balloon, though other anchors could be used, including other balloon shapes. In FIG. 12F the distal end has been positioned within the region of the lung (bronchial region 1212) that is to be treated (so that it is in fluid communication with the downstream region that is collapsed), and the anchor 1270 may be deployed to expand and secure the distal end region of the apparatus within the bronchial region 1212 as shown.

Once secured in place, as shown in FIG. 12G, the inner member 1241, which in this example includes a camera, a pair of lights and two working channels, may be withdrawn from the large bore lumen 1240 of the outer member 1210. The outer member includes a pressure sensor 1211 as described above. Thus, in FIG. 12H, the apparatus includes just the outer member and access to the region distal of the anchor is provided through the outer member.

In some examples the controller may control the application of pressure as the inner member is withdrawn; optionally the inner member may be withdrawn without applying pressure (e.g., air), as the displaced volume when removing the inner member may be sufficiently small and/or the anchor may allow passage of air through or around the anchor.

In use, once positioned the apparatus may apply ventilation though the large bore of the outer member and may initially apply air pressure to reinflate the collapsed region of the lung 1251′ as illustrated in FIGS. 121 and 12J. In FIG. 121, the distal end of the apparatus 1200 may be anchored in position and positive pressure may be applied, resulting in reinflation of the collapsed lung region 1251′ as shown in FIG. 12J. Ventilation may be applied through the large bore lumen.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

FIGS. 13A-13B illustrate another example, similar to that shown in FIGS. 12A-12J, using an outer member that is configured as a sheath for use with a commercially available inner member (e.g., endoscope, such as a bronchoscope). For example, in FIG. 13A, the distal end of the apparatus has been advanced within a bronchus 1312 and the anchor 1370 has been expanded. In this example the anchor 1370 is an annular balloon, though other anchors could be used, including other balloon shapes. The apparatus has been positioned with the inner member (e.g., bronchoscope) 1341 inserted. Thus, the distal end has been positioned within the region of the lung (bronchial region 1312) that is to be treated, and the anchor 1370 is deployed to secure the distal end region of the apparatus within the bronchial region as shown.

The inner member 1341, which may include a camera, a pair of lights and two working channels, may be withdrawn from the large bore lumen of the outer member (sleeve) 1310. The outer member (sleeve) includes a pressure sensor 1311 as described above. Thus, in FIG. 13B, the apparatus includes just the outer member and access to the region distal of the anchor is provided through the sleeve. The inner lumen may therefore act as a working channel. The apparatus may otherwise be used as described above. Thus, once the internal endoscope (e.g., bronchoscope) is removed, what remains in place is a large working channel that allows for all kind of different therapeutic uses in selective areas of the lungs. Thus, the apparatuses described herein may be used with virtually any flexible instrument that fits into the lumen (e.g., working channel) and therefore can be delivered to the lung periphery. The methods and apparatuses described herein allow for the delivery of the working channel under direct visualization and the anchoring onto a desired position.

In any of the methods and apparatuses described herein, the outer member (e.g., sheath) may include a plurality of sensors in the distal end region, including at or near the distal face. The inner member (e.g., endoscope) may include no or separate sensors. The outer member may include a variety of different types of sensors, including one or more pressure sensors, thermal sensors, cameras, etc. For example, FIG. 13C illustrates an example of a system including an outer (sheath) member that is configured to seal off a portion of the lungs and form a passage for an inner member (e.g., bronchoscope), similar to that shown in FIGS. 12A-12J and FIGS. 13A-13B. In this example the outer member may also be configured as a sheath for use with a commercially available inner member (e.g., endoscope, such as a bronchoscope). In FIG. 13C, the distal end of the apparatus has been advanced within a bronchus 1312 and the anchor 1370 (sealing anchor) has been expanded. The anchor 1370 is shown as an annular balloon, though other sealing anchors could be used, including other balloon shapes. As in any of these apparatuses the sealing anchor may be configured to form a seal with the bronchus without damaging or irritating the walls of the bronchus. In FIG. 13C the apparatus has been positioned with the inner member (e.g., bronchoscope) 1341′ inserted. Thus, the distal end has been positioned within the region of the lung (bronchial region 1312) that is to be treated, and the anchor 1370 is deployed to secure and seal the distal end region of the apparatus within the bronchial region. The sleeve shown also include a plurality of different sensors, including a pressure sensor 1311′ and multiple additional sensors 1312, 1312″. Any of these sensors may be optical sensors (e.g., cameras, visible light cameras/sensors, UV light cameras/sensors, near-IR light cameras/sensors, specific wavelength sensors, etc.), thermal sensors, force sensors, chemical sensors, etc. One or more light sources may also be included on the sheath (in FIG. 13C, for example a light source output 1312′ may be included.

In FIG. 13C, the inner member 1341′, which may also include one or more sensors (not shown), such as a camera, lights and/or one or more working channels. In some examples the inner member may be simplified, and may not include a sensor, but may include one or more working channels and/or a manipulator.

The inner member 1341′ may be withdrawn from the large bore lumen of the outer member (sleeve) 1310′. The apparatus may include the outer member, and access to the region distal of the anchor is provided through the sleeve. The inner lumen may therefore act as a working channel. The apparatus may otherwise be used as described above. Thus, once the internal endoscope 1341′ (e.g., bronchoscope) is removed, what remains in place is a large working channel that allows for all kind of different therapeutic uses in selective areas of the lungs. This is illustrated in FIG. 13D. The sleeve apparatuses described herein may be used with virtually any flexible instrument that fits into the lumen (e.g., working channel) and therefore can be delivered to the target lung region. The methods and apparatuses described herein allow for the delivery of the working channel under direct visualization and the anchoring onto a desired position. In FIG. 13D the inner member (e.g., scope, such as an endoscope) is retracted and/or removed, leaving the outer sleeve in position.

FIGS. 13C and 13D show an end view of an example of an apparatus including an outer member (sleeve) and an inner member (e.g., endoscope or inner sleeve). The outer member may be a multi-function endobronchial tube and may include a large lumen, channel, or bore in which the inner member may engage. The outer member may include pressure sensor, which may include a lumen (pressure sensing lumen). As mentioned, the outer member can also include other sensors such as video, light temperature or energy sensors.

The methods and apparatuses described herein may work particularly well for solid organs, including the lungs. These apparatuses may allow for a local delivery of an agent (e.g., gas, liquid, semi-solid, e.g., gel, and/or solid) for delivering treatment, including treatment for lung cancer, or other disorder. In general, the outer sheath may be positioned within the organ and may be secured in place to form a seal to prevent material to be delivered from extending proximally past the distal end region of the apparatus.

Thus, as mentioned above, any of these apparatuses may be configured to include a sealing region (e.g., a seal) that prevents a liquid, gas, and/or solid material. Further, this sealing region may be configured to prevent damage to the wall(s) of the lumen into which the apparatus is inserted and anchored (e.g., bronchus). In some examples the sealing anchor may be an expandable/inflatable member such as a balloon or may be a mechanically expandable member that include an outer sealing or cover surface. The anchor may extend along the length of the distal end region to allow sealing (e.g., may extend between 1 mm and 12 cm, between 1 cm and 10 cm, between 1 cm and 8 cm, between 1 cm and 6 cm, between 1 cm and 5 cm, between 1.5 cm and 15 cm, between 1.5 cm and 12 cm, between 1.5 cm and 10 cm, between 1.5 cm and 5 cm, between 2 cm and 15 cm, between 2 cm and 12 cm, between 2 cm and 10 cm, between 2 cm and 8 cm, between 2 cm and 6 cm, etc.). The outer sealing surface may include one or more ridges, extending radially and/or longitudinally. The outer sealing surface may be coated with a material, such a hydrophilic coating.

In any of these examples the sealing anchor maybe configured to limit the expansion of the sealing member to prevent damage to the walls of the lumen within he lungs. The sealing anchor may be configured to limit expansion by including feedback (e.g. pressure feedback) to prevent expansion if the pressure is increasing faster than a predetermined value (indicating contact with the wall(s) of the lumen). In some cases the sealing anchor may be configured to limit expansion to a percentage of the un-expanded diameter (e.g., 5%, 10%, 15%, 25%, 30%, 35%, 40%, 45%, 50%, 60%, etc.). In general, because the anchors described herein may allow for sealing and isolation of a selected region (e.g., segment) of the lungs, they may permit controlled local application of a therapeutic material, as described above.

In any of these apparatuses the sealing anchor may be reinforced (e.g., radially and/or longitudinally reinforced).

In any of these apparatuses the outer sleeve (outer member) may be steerable. Alternatively and/or additionally (and in some examples, preferentially) the inner member may be steerable. For example, the inner member may be advanced independently of the outer member (e.g., inch-wormed out of the outer member) and steered through the lumen, into one or more specific branches, periodically advancing the outer member over the inner member to follow the path formed by the inner member.

Any of these apparatuses may be used for local ventilation and/or pressurization (ad described above), and/or for local treatment, including the application of one or more agents (e.g., gas, vapor, gel, etc.). Agents may include liquids, which may be nebulized to a desired particle range, gas, etc. Agents may include one or more active agents, such as drugs. Thus, these apparatuses may be used to locally apply one or more agents for local treatment. Agents may be applied through the outer member (sleeve), including through the large-bore inner lumen or one or more accessory lumen.

Any of the apparatuses and methods described herein may be used in multiples to concurrently treat many different and/or overlapping regions of the lung. For example, FIG. 14 illustrates an example in which multiple apparatuses are inserted into the patient's lungs 1402. Each apparatus 1409, 1409′, 1409″, 1409′, 1409′ in this example (five are shown) may be inserted through the trachea, including from the mouth or oral cavity. In FIG. 14 they are inserted in tandem (side-by-side) and into different branches (bronchi) of the lungs. Proximally, the handles for each of the apparatuses may be configured to engage with each other and each of the outer members (e.g., sleeves) may be coupled to the same controller, or some or all of them may be coupled to different controllers. It may be particularly beneficial to couple all of the sleeves to the same controller, so that the controller may coordinate the pressure (including the application of respiration through each sleeve). In some examples the controller may therefore have multiple operational modes (ventilation modes) for controlling each of the different apparatuses inserted into the lungs.

Thus, these methods and apparatuses may allow for the individual and selective isolation of different regions of the lungs. Any number of apparatuses may be used (e.g., one, two, three, four, five, etc.), allowing independent ventilation and/or treatment, as described above. The different apparatuses may be inserted individually (e.g., one by one) or collectively.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed, which may provide non-uniform (e.g., regional) ventilation.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively, or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is ±0.1% of the stated value (or range of values), ±1% of the stated value (or range of values), ±2% of the stated value (or range of values), ±5% of the stated value (or range of values), ±10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A pulmonary isolation and ventilation system comprising:
an outer member comprising a flexible endobronchial tube having a lumen extending therethrough and a pressure sensor at a distal end;
an elongate inner member configured to sealingly fit within the lumen;
a first handpiece portion coupled to the outer member;
a second handpiece portion coupled to the inner member and configured to releasably engage the first handpiece portion; and
a controller configured to couple to the first handpiece portion and to receive input from the pressure sensor, and to couple to the second handpiece portion.

2. The system of claim 1, wherein the controller is further configured to regulate a pressure at the distal end of the outer member when the elongate inner member is withdrawn from the lumen of the outer member.

3. The system of claim 1, wherein the elongate inner member comprises one or more cameras and one or more light sources.

4. The system of claim 1, wherein the controller is configured to apply respiration through the lumen when the elongate inner member is removed from the lumen.

5. The system of claim 1, further comprising a nebulizer configured to administer a therapeutic from the outer member.

6. The system of claim 1, further comprising an inflatable anchor at a distal end region of the outer member.

7. The system of claim 1, further comprising a pressure channel extending through the elongate inner member, wherein the controller is configured to apply positive or negative pressure through the pressure channel to regulate the pressure at the distal end of the outer member when the elongate inner member is withdrawn from the lumen of the outer member.

8. The system of claim 1, wherein the outer member is steerable.

9. A pulmonary isolation and ventilation system comprising:
an outer member configured as a sheath comprising a flexible endobronchial tube having a lumen extending therethrough and a one or more sensors at a distal end, the outer member comprising an elongate inner lumen extending from the distal end to a proximal end, and a sealing anchor configured to extend from an outer diameter of a distal end region of the outer member to seal the outer member within a bronchial lumen; and
a handpiece portion coupled to the outer member; and
a controller configured to couple to the handpiece portion and to receive input from the one or more sensors.

10. The system of claim 9, wherein the elongate inner lumen comprises more than 65% of the cross-sectional area of the outer member.

11. The system of claim 9, further comprising an elongate inner member configured to fit within the elongate inner lumen and to engage the handpiece.

12. The system of claim 9, wherein the controller is further configured to regulate a pressure at the distal end of the outer member.

13. The system of claim 9, wherein the one or more sensors comprises one or more cameras and wherein the outer member comprises one or more light sources.

14. The system of claim 9, wherein the one or more sensors comprise one or more of: visualization sensors, illumination sensors, temperature sensors, or pressure sensors.

15. The system of claim 9, wherein the sealing anchor comprises an expandable balloon.

16. The system of claim 9, wherein the controller is configured to apply control the sealing of the sealing anchor.

17. The system of claim 9, further comprising a nebulizer configured to administer an agent from the lumen of the outer member.

18. A method comprising:
inserting an outer member comprising a flexible endobronchial tube having a lumen extending therethrough into a region of a patient's lung, wherein an elongate inner member is sealed within a lumen of the outer member and extends to a distal end region of the outer member;
confirming placement of a distal end of the outer member withing the lung;
sealingly anchoring the distal end of the outer member within the lung to form a sealed-off region of the lung;
withdraw the elongate inner member from the lumen of the outer member; and
delivering an agent comprising one or more of: a drug, a hydrogel, a gel, particles, nanoparticles, liposomes, cells, stem cells, vesicles, or gene therapy through the lumen and into the sealed-off region of the lung.

19. The method of claim 18, wherein sealing anchoring the distal end of the outer member comprises controlling the application of a pressure to expand a sealing anchor from an outer diameter of the outer member.

20. The method of claim 18, further comprising controlling the application of pressure to regulate a pressure at the distal end of the outer member as the elongate inner member is withdrawn from the lumen of the outer member.

21. The method of claim 20, wherein controlling pressure comprises preventing a vacuum at the distal end of the outer member.

22. The method of claim 18, wherein confirming placement comprises visualizing the region of the lung near the distal end of the outer member using one or more cameras of the inner member.

23. The method of claim 18, further comprising disengaging a first handle portion that is coupled to the inner member from a second handle portion that is coupled to the outer member prior to withdrawing the inner member from the lumen of the outer member.

24. The method of claim 18, further comprising applying an atomized drug from a distal end of the outer or inner member.
